(19) United States
(12) Patent Application Publication
(10) Pub. No.: US 2013/0296976 A1
(43) Pub. Date: Nov. 7, 2013

(54) DYNAMIC WAVELENGTH ADAPTING DEVICE TO AFFECT PHYSIOLOGICAL RESPONSE AND ASSOCIATED METHODS

(71) Applicants: Fredric S. Maxik, Indialantic, FL (US); David E. Bartine, Cocoa, FL (US); Robert R. Soler, Cocoa Beach, FL (US)

(72) Inventors: Fredric S. Maxik, Indialantic, FL (US); David E. Bartine, Cocoa, FL (US); Robert R. Soler, Cocoa Beach, FL (US)

(73) Assignee: LIGHTING SCIENCE GROUP CORPORATION, Satellite Beach, FL (US)

(21) Appl. No.: 13/465,781

(22) Filed: May 7, 2012

(51) Int. Cl. A61N 5/06

(52) U.S. Cl. 607/88

(57) ABSTRACT

A light converting device is described for receiving source light within a source wavelength range, converting the source light into an interim light, and converting the interim light into a converted light. The lighting device may include an enclosure with an application of a wide production conversion coating and a narrow production conversion coating to perform a series of wavelength conversion operations on a source light to produce a converted light.

### FIELD OF THE INVENTION

The present invention relates to the field of wavelength conversions for lighting devices and, more specifically, to dynamically selectable wavelength conversions to convert light to affect a physiological response.

### BACKGROUND OF THE INVENTION

Displays are widely used in our lives to present images, video, documents, or other content to a viewer. As the display has grown into larger roles within our lives, there has been a need to improve the technology that powers the display.

For many years, displays have relied on the cathode ray tube (CRT) to provide a succession of rapidly refreshing images to a user. The CRT operated by including phosphors and an electron gun within a vacuum tube. Typically, the vacuum tube was constructed from glass, making the surround display bulky, heavy, and fragile.

Upon the advent of alternative display technologies, such as plasma screens and liquid crystal displays (LCD), the size and bulk previously associated with the display had been reduced. These newer and more compact screens allowed the display of a high resolution image using a device with a significantly reduced spatial and power consumption footprint.

As the small format display continued to evolve, the formerly passive-matrix LCD screens were largely replaced with active-matrix LCD screen. As the active-matrix LCD became more ubiquitous, combined with improved backlighting, brighter and sharper images could be displayed to a user. The technology that drives backlighting has also evolved. Formerly fluorescent tube backlights dominated the compact screens. Now, alternate lighting technologies, such as light emitting diodes (LEDs) are becoming more commonly used in backlights.

However, LEDs and other lighting technologies may emit a concentration of light in a wavelength range that may have undesired affects on the physiological responses of humans and other organisms. One such physiological response that may be affected by the emission of lighting within a specific wavelength range includes the production of chemicals that control the circadian rhythm of an organism, such as melatonin. Previous attempts to alter the inclusion of light within the affective wavelength range have used filters to remove the light, resulting in decreased light output and thus decreased efficiency. Additionally, previous attempts to alter the inclusion of light within the affective wavelength range disclose permanent solutions, or solutions that are not easily, readily, or dynamically adjustable.

There exists a need for an apparatus that provides an ability to receive a light emitted from a light source with a first level of a biological affective wavelength range and convert the source light into a converted light with a second level of the biological affective wavelength range. There further exists a need for wavelength conversion operation to be performed relative to a user input, sensory information, or other dynamic stimulus, as may be determined by a controller.

### SUMMARY OF THE INVENTION

With the foregoing in mind, the present invention is related to an apparatus that provides an ability to receive a light emitted from a light source with a first level of a biological affective wavelength range and convert the source light into a converted light with a second level of the biological affective wavelength range. The apparatus of the present invention may additionally perform the wavelength conversion operation relative to a user input, sensory information, or other dynamic stimulus, as may be determined by a controller.

By providing a light converting device that advantageously performs a wide and narrow production light conversion operation, away from the heat generating light source, the present invention may beneficially possess characteristics of reduced complexity, size, and manufacturing expense.

These and other objects, features, and advantages according to the present invention are provided by a wavelength converting device for adapting light that includes a source light. The wavelength converting device may include a wavelength conversion material to convert the source light into a converted light to be included generally in the light. The source light may include a first level of affective light within a biological affective wavelength range that affects a physiological response. The converted light may include a second level of the affective light within the biological affective wavelength range. The converting device may also include a controller to control operation between a normal mode and an altered mode. The normal mode may be defined by the second level of the affective light being substantially similar to the first level of the affective light. The altered mode may be defined by the second level of the affective light differing from the first level of the affective light.

The source light including the first level of the affective light may be defined by a first chromaticity. The converted light including the second level of the affective light may be defined by a second chromaticity. The first chromaticity may be substantially similar to the second chromaticity.

The altered mode may include an increased mode and a decreased mode. The increased mode may be defined by the second level being greater than the first level, and the decreased mode may be defined by the second level being less than the first level. The physiological response may be melatonin production, and the light may be emitted from a light source. In some embodiments of the present invention, the light source may include a light emitting diode (LED).

More specifically, the light source may include a non-affective light source and an affective light source. The non-affective light source may emit the source light with the first level of the affective light, and the affective light source may include the wavelength conversion optic to emit the source light and convert the source light into the converted light with the second level of the affective light. The affective light source and the non-affective light source may advantageously be selectively enabled.

The wavelength conversion material may be carried by a selectively rotatable disc to enable operation between the normal mode and the altered mode. The rotatable disc may include a plurality of portions. Each of the plurality of portions may correlate with at least one condition. The rotatable disc may be positionable to selectively receive the light at each portion to manipulate the light. The conditions may be color, biological affect, chromaticity, luminosity, saturation, and/or hue.

The wavelength converting device may include a mirror having a light reflective surface to receive and reflect the light. The wavelength conversion material may be located adjacent to at least part of the light reflective surface. The source light may be received by the mirror during the altered mode, and may be converted by the wavelength conversion material to the converted light with the second level of the affective light to be reflected. The wavelength conversion material may be located adjacent to a first part of the reflective surface. In some embodiments of the present invention, no wavelength conversion material may be located adjacent to a second part of the reflective surface, and the first part of the reflective surface may receive and converts the source light to the converted light to be reflected with the second level of affective light. The second part of the reflective surface may receive the source light to be reflected with the first level of affective light. The mirror may be included in an array of mirrors, and reflection of the light from each mirror in the array of mirrors may be selectable.

The mirror may be a repositionable mirror to be selectively repositioned by the controller. The repositionable mirror may be included in an array of repositionable mirrors. The wavelength conversion material may be located adjacent to at least one repositionable mirror included in the array to receive and convert the source light to the converted light to be reflected with the second level of affective light. In some embodiments of the present invention, approximately no conversion material may be located adjacent to at least one repositionable mirror included in the array to receive the source light to be reflected with the first level of affective light. The repositionable mirror may be included in a microelectromechanical device (MEMS).

The wavelength converting device may also include a sensor to detect ambient light and generate ambient level information to be communicated to the controller regarding the ambient light. The controller may analyze the ambient level information to control operation between the normal mode and the altered mode. The wavelength converting device may also include a sensor to detect a spectral content of ambient light and generate spectral information to be communicated to the controller regarding the spectral content of the ambient light. The controller may analyze the spectral information to control operation between the normal mode and the altered mode. In other embodiments of the present invention, the wavelength converting device may include a timer to generate timer information to be communicated to the controller regarding a time period. Accordingly, the controller may analyze the timer information to control operation between the normal mode and the altered mode.

The controller may be communicatively connected to a radio logic board to transmit and receive communication information using a network. The communication information may be used by the controller to control operation between the normal mode and the altered mode. The biological affective wavelength range may be defined as being essentially between 460 nanometers and 490 nanometers. The brightness of the source light and the converted light may be controllable by the controller. The source light may be received by a display. In some embodiments, the display may be a liquid crystal display (LCD). The display may use color field sequential switching, and the display may be included in a computerized device.

A method aspect of the present invention is for adapting light that includes a source light using a wavelength converting device. The method may include operating the wavelength converting device between a normal mode and an altered mode, as described above.

### BRIEF DESCRIPTION OF THE DRAWINGS

FIG. 1 is a schematic diagram of a wavelength adapting device in use according to an embodiment of the present invention.

FIG. 2 is a block diagram of a controller for use in an embodiment of the present invention.

FIG. 3 is a schematic partial elevation view of an array of light sources and wavelength conversion material used in connection with a wavelength adapting device according to an embodiment of the present invention.

FIG. 4 is a schematic diagram illustrating the array of light sources and wavelength conversion material shown in FIG. 3.

FIG. 5 is a schematic diagram illustrating an array of increasing, decreasing, and normal light sources used in connection with a wavelength adapting device according to an embodiment of the present invention.

FIG. 6 is a schematic block diagram of a wavelength adapting device that allows moveable positioning of wavelength conversion material according to an embodiment of the present invention.

FIG. 7 is a schematic block diagram of a wavelength adapting device in use with a first mirror and a second mirror having a conversion coating according to an embodiment of the present invention.

FIG. 8 is a schematic block diagram of a wavelength adapting device in use with two mirrors and a third mirror having a conversion coating according to an embodiment of the present invention.

FIG. 9 is a schematic diagram illustrating an LED array having a plurality of wavelength conversion coatings according to an embodiment of the present invention.

FIG. 10 is a graphical depiction of a waveform of a source light of a wavelength adapting device according to an embodiment of the present invention.

FIG. 11 is a graphical depiction of a waveform of a color converted light of a wavelength adapting device according to an embodiment of the present invention.

FIG. 12 is a graphical depiction of a waveform of a converted light of a wavelength adapting device having a broad high energy wavelength range according to an embodiment of the present invention.

FIG. 13 is a graphical depiction of a waveform of a converted light of a wavelength adapting device having a broad low energy wavelength range according to an embodiment of the present invention.

FIG. 14 is a graphical depiction of a waveform of a converted light of a wavelength adapting device having a narrow high energy wavelength range according to an embodiment of the present invention

FIG. 15 is a graphical depiction of a waveform of a converted light of a wavelength adapting device having a narrow wavelength range according to an embodiment of the present invention.

FIG. 16 is a graphical depiction of a waveform of a converted light of a wavelength adapting device having a narrow wavelength range according to an embodiment of the present invention.

FIG. 17 is a schematic diagram of a wavelength adapting device in use with a screen according to an embodiment of the present invention.

FIG. 18 is a schematic diagram of a wavelength adapting device in use with a plurality of repositioning mirrors according to an embodiment of the present invention.

FIG. 19 illustrates a plurality of alternate rotatable conversion materials for use with a wavelength adapting device according to an embodiment of the present invention.

FIG. 20 is a schematic diagram of a wavelength adapting device in use with a plurality of repositioning mirrors and a plurality of conversion materials according to an embodiment of the present invention.

FIG. 21 is a schematic diagram of a wavelength adapting device in use with a plurality of repositioning mirrors and a rotatable conversion material according to an embodiment of the present invention.

FIG. 22 is an illustration of an alternate configuration of a wavelength adapting device using a repositionable sheet having a conversion material thereon about a light source according to an embodiment of the present invention.

FIGS. 23-28 are flowcharts depicting embodiments of operation of a wavelength adapting device according to the present invention.

### DETAILED DESCRIPTION OF THE PREFERRED EMBODIMENT

The present invention will now be described more fully hereinafter with reference to the accompanying drawings, in which preferred embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. Those of ordinary skill in the art realize that the following descriptions of the embodiments of the present invention are illustrative and are not intended to be limiting in any way. Other embodiments of the present invention will readily suggest themselves to such skilled persons having the benefit of this disclosure. Like numbers refer to like elements throughout.

In this detailed description of the present invention, a person skilled in the art should note that directional terms, such as “above,” “below,” “upper,” “lower,” and other like terms are used for the convenience of the reader in reference to the drawings. Also, a person skilled in the art should notice this description may contain other terminology to convey position, orientation, and direction without departing from the principles of the present invention.

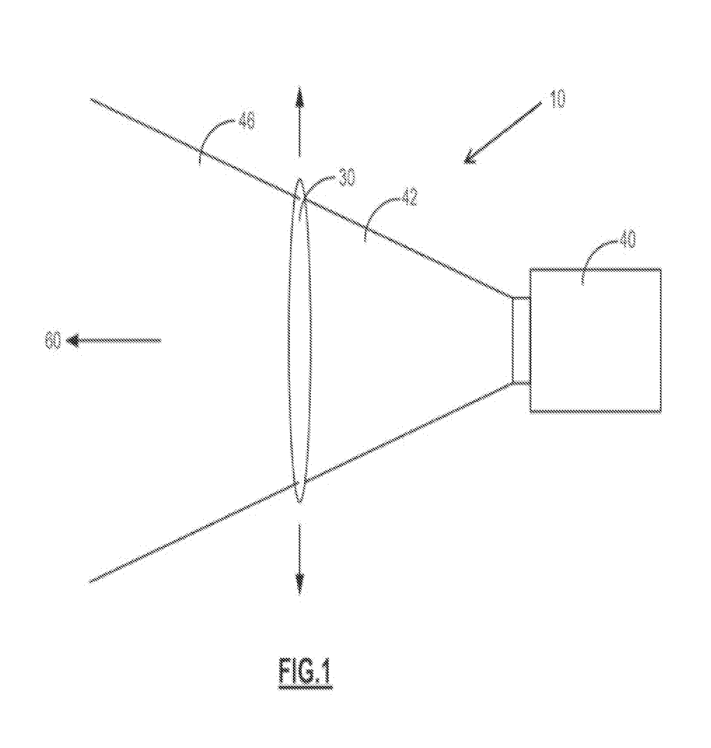

Referring now to FIGS. 1-22, a wavelength adapting device 10 according to the present invention is now described in greater detail. Throughout this disclosure, the wavelength adapting device 10 may also be referred to as a device, optic, system or the invention. Alternate references of the wavelength adapting device 10 in this disclosure are not meant to be limiting in any way.

As perhaps best illustrated in FIG. 1, the wavelength adapting device 10, according to an embodiment of the present invention, may include a wavelength conversion material 30 to receive a source light 42 and convert the source light 42 into a converted light 46. An enclosure 50 may be included to receive a source light 42. The enclosure 50 may include a first level of affective light, defined within a biological affective wavelength range. The source light 42 may be converted to a converted light 46 within a second level of affective light in the biological affective wavelength range. The converted light 46 may be directed to a desired output direction 60. The wavelength adapting device 10 may additionally include one or more mirror 33.

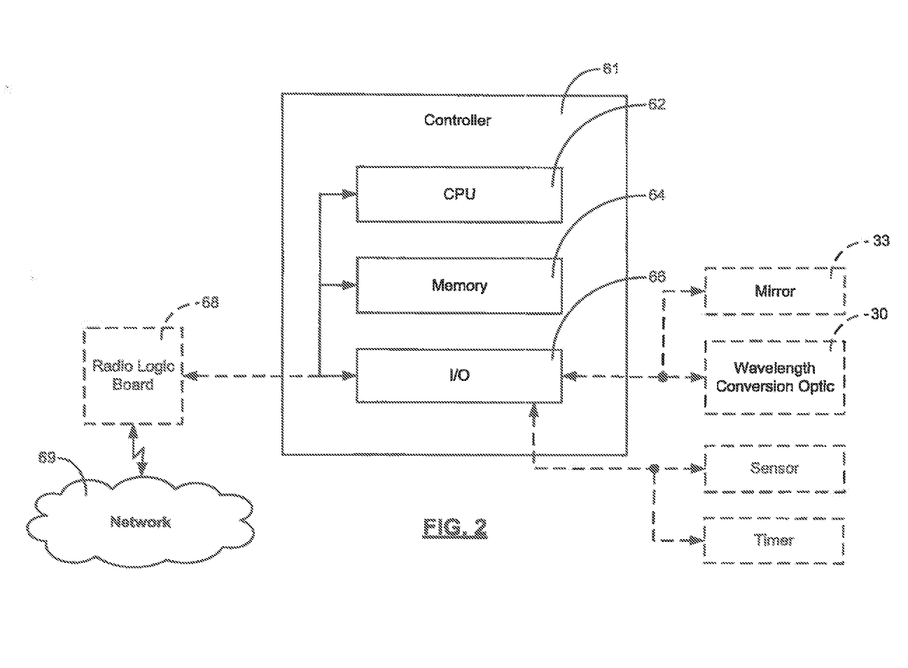

The wavelength adapting device 10, according to an embodiment of the present invention, may include a controller 61 to control the wavelength conversion operation. Referring to FIG. 2, the controller 61 will now be discussed in greater detail. The controller 61 may include a CPU 62, memory 64, and an input/output (I/O) interface 66. The CPU 62 may compute and perform calculations to the data received by additional components, such as, for example, sensors. The CPU 62 may receive feedback information from timers, sensors, user input, and other components. Feedback information may include ambient light or spectral content sensing element. Also, the CPU 62 may analyze the data received by the sensors, timer, user input, or other component to control the operation of the wavelength adapting device 10 of the present invention.

The controller 61 may also include memory 64. The memory 64 may include volatile and non-volatile memory modules. Volatile memory modules may include random access memory (RAM), which may temporarily store data and code being accessed by the CPU 62. The non-volatile memory 64 may include flash based memory 64, which may store the computerized program that may be operated on the CPU 62 and sensory data that may be received by the sensors and other components during operation of the wavelength adapting device 10.

Additionally, the memory 64 may include computerized code used by the CPU 62 to control the operation of the wavelength adapting device 10. The memory 64 may also store feedback information relating to the operation of additional components included in, or interfacing with, the wavelength adapting device 10. Furthermore, the memory 64 may include an operating system, which may additionally include applications that may be run from within the operating system, as would be appreciated by a person of skill in the art.

The controller 61 may additionally include an I/O interface 66. The I/O interface 66 may control the receipt and transmission of data between the controller 61 and additional components. Provided as a non-limiting example, the I/O interface 66 may receive a data communication signal from sensors and/or timers. After the CPU 62 has analyzed the system, the I/O interface 66 may transmit a control signal to a component. The control signal may be used to modify the position of a wavelength conversion material 30, mirror 33, or other element. This modification of position may be performed by using, for example, an electromechanical system.

An electromechanical system may be defined as a system that converts electrical energy into mechanical motion. As an example, an electromechanical system may receive a signal from the controller 61. The electromechanical system may convert the electrical signal into a controlled physical motion. More specifically, the electromechanical system may generate the physical motion via a piston, rotating member, motor, servo-actuator, electric attraction and/or repulsion, or other electrically powered motion generating device.

Electrical signals may include various signal characteristics, which may result in various corresponding physical motions performed by the electromechanical system. The electrical signal may be digital, which may transmit a control signal from the controller 61 that may be interpreted by the electromechanical system. The electromechanical system may then generate the physical motion in response to the interpreted digital signal. Alternately, the electrical signal may be analog, which may transmit a varied voltage or current. The varied voltage or current transmitted in the analog signal may be used to control the amount of physical motion created by the electromechanical system. A person of skill in the art will appreciate additional control signals to be included within the scope and spirit of the present invention.

The controller 61 may additionally be operatively connected to a radio logic board 68, through which the controller 61 may communicate with additional devices using a network 69. More specifically, the controller 61 and the radio logic board 68 may be connected, for example, through the I/O interface 66 included within the controller 61. A person of skill in the art will appreciate additional locations for the radio logic board 68, such as being included within the controller 61, to allow the radio logic board 68 to communicate with a network 69 as being included within the scope of the present invention. The radio logic board 68 may allow the controller 61 to communicate with additional electronic devices, such as a computerized device, mobile computing device, or remotely located controller 61.

The radio logic board 68 may additionally be operatively connected to one or more antenna. Data may be included in a communication signal, which may be broadcasted and/or received by the radio logic board 68 through the antenna, and thus be communicated with the controller 61. A person of skill in the art will appreciate that the radio logic board 68 may communicate with a network connected device via a wired and/or wireless network 69. A wireless network 69 may include, but should not be limited to, a radio network, infrared network, or other wireless communication network 69.

The memory 64 of the controller 61 may be programmed or manipulated by an external device over the network 69. The programming or manipulation of the memory 64 may, for example and without limitation, allow the wavelength adapting device 10 of the present invention to alter a plurality of parameters, such as sensitivity of an included sensor, timing settings of an included timer, or the level that the wavelength adapting device 10 may increase or decrease affective light to be included within a converted light 46. The inclusion of a radio logic board 68 in an electronic lighting device 10 has been described in greater detail in U.S. Patent Application 61/486,314 to Maxik, et al., the entire contents of which is incorporated herein by reference.

The sensor, communicatively connected to the controller 61, may receive and detect the presence or intensity of a condition and transmit the detected condition to the controller 61 as information. The communicative connection may be wired or wireless. Examples of a condition may include, but should not be limited to, a user initiated action, ambient light levels, ambient light chromaticity, time, or duration. More specifically, an ambient light sensor may detect the current level of ambient light in a location in which illumination is being affected by the wavelength adapting device 10, a remote location, or virtually any other location. The controller 61 may receive the ambient light information from the ambient light sensor, which may be analyzed to control the operation of the wavelength adapting device 10 between the normal mode and the altered mode. Thus, the controller 61 may advantageously adapt the levels of affective light included in the converted light 46 in response to the ambient light levels of light within a given environment.

An additional example of a sensor communicatively connected to the controller 61 may include a spectral content sensor to analyze the spectral content of light. The light that may be analyzed by the spectral content sensor may be in the location in which illumination is being affected by the wavelength adapting device 10 of the present invention, a remote location, or virtually any other location. The spectral content of light may include spectral information regarding the intensity of light in one or more specific wavelength range. The controller 61 may receive the spectral information from the spectral content sensor, which may be analyzed to control the operation of the wavelength adapting device 10 between the normal mode and the altered mode. Thus the controller 61 may advantageously adapt the levels of affective light included in the converted light in response to the spectral content of light within a given environment.

Referring back to FIG. 1, a light source 40, which may emit a source light 42, will now be discussed. As illustrated, for example, in FIG. 1, the wavelength conversion material 30 may receive the source light 42, which may be originated from a light source 40. In the present invention, the light source 40 may include light emitting diodes (LEDs) capable of emitting light that may include a first level of affective light. Affective light may be defined as light with levels of intensity within a biological affective wavelength range. The biological affective wavelength range will be discussed in greater detail below. Additional embodiments of the present invention may include a source light 42 that is generated by a laser driven light source 40. Those skilled in the art will appreciate that the source light 42 may be provided by any number of lighting devices, and may include varying levels of affective light. A skilled artisan will additionally appreciate that, although the light source 40 is described as using a light emitting semiconductor throughout this disclosure, any light generating structure may be used and remain within the scope and spirit of the present invention.

An LED may emit light when an electrical current is passed through the diode. The LED may be driven by the electrons of the passing electrical current to provide an electroluminescence, or emission of light. The color of the emitted light may be determined by the materials used in the construction of the light emitting semiconductor. The foregoing description contemplates the use of semiconductors that may emit a light in the blue or ultraviolet wavelength range. However, a person of skill in the art will appreciate that light may be emitted by light emitting semiconductors of any wavelength range and remain within the breadth of the invention as disclosed herein. Effectively, a light emitting semiconductor may emit a source light 42 in any wavelength range, since the emitted source light 42 may be subsequently converted by a wavelength conversion material 30 as it is reflected and/or directed in the desired output direction 60.

As previously mentioned, the source light 42 may be emitted in blue or ultraviolet wavelength ranges. Additionally, a portion of the light within the blue wavelength range may be within a biological affective wavelength range, which may affect the physiological responses of an organism. This blue light within the biological affective wavelength range may be classified as affective light.

However, a person of skill in the art, after having the benefit of this disclosure, will appreciate that LEDs capable of emitting light in any number of wavelength ranges may be used in the light source 40, in accordance with this disclosure of the present invention. A skilled artisan will also appreciate, after having the benefit of this disclosure, additional light generating devices that may be used in the light source 40 that are capable of creating an illumination.

The present invention may include a light source 40 that generates source light 42 that includes light with wavelengths in the blue spectrum. The blue spectrum may include light with a wavelength range between 400 and 500 nanometers. A source light 42 in the blue spectrum may be generated by a light emitting semiconductor comprised of materials that may emit a light in the blue spectrum. Examples of such light emitting semiconductor materials may include, but are not intended to be limited to, zinc selenide (ZnSe) or indium gallium nitride (InGaN). These semiconductor materials may be grown or formed on substrates, which may be comprised of materials such as sapphire, silicon carbide (SiC), or silicon (Si). A person of skill in the art will appreciate that, although the preceding semiconductor materials have been disclosed herein, any semiconductor device capable of emitting a light in the blue spectrum is intended to be included within the scope of the present invention.

Additionally, the present invention may include a light source 40 that generates source light 42 that includes light with wavelengths in the ultraviolet spectrum. The ultraviolet spectrum may include light with a wavelength range between 200 and 400 nanometers. A source light 42 in the ultraviolet spectrum may be generated by a light emitting semiconductor comprised of materials that may emit a light in the ultraviolet spectrum. Examples of such light emitting semiconductor materials may include, but are not intended to be limited to, diamond (C), boron nitride (BN), aluminum nitride (AlN), aluminum gallium nitride (AlGaN), or aluminum gallium indium nitride (AlGaInN). These semiconductor materials may be grown or formed on substrates, which may be comprised of materials such as sapphire, silicon carbide (SiC), or Silicon (Si). A person of skill in the art will appreciate that, although the preceding semiconductor materials have been disclosed herein, any semiconductor device capable of emitting a light in the ultraviolet spectrum is intended to be included within the scope of the present invention.

A previously mentioned, the source light 42 may be generated by a light source 40 to include affective light within a biological affective wavelength range. More specifically, in an example wherein the physiological affect affected by the affective light is melatonin production, the biological affective wavelength range may include light with a wavelength range between 460 and 490 nanometers. A source light 42 including affective light may be generated by a light emitting semiconductor device comprised of materials that may additionally emit a light in the blue spectrum, as has been discussed above. A person of skill in the art will appreciate that the varying combination of semiconductor materials and their application may result in source light being emitted by the light source with varying levels of light within the biological affective wavelength range. A skilled artisan will understand the varying levels to include, and vary between, a high level and a low level. Light within the biological affective range may induce or inhibit the physiological response from a human or other organism physiological, which will be discussed in greater detail below.

The light source 40 of the present invention may include an organic light emitting diode (OLED). An OLED may be a comprised of an organic compound that may emit light when an electric current is applied. The organic compound may be positioned between two electrodes. Typically, at least one of the electrodes may be transparent. An OLED may additionally emit a source light that includes light within the biological affective wavelength range.

A person of skill in the art will appreciate that the wavelength adapting device 10 may receive a source light 42 that is monochromatic, bichromatic, or polychromatic. A monochromatic light is a light that may include one wavelength range. A bichromatic light is a light that includes two wavelength ranges that may be derived from one or two light sources 40. A polychromatic light is a light that may include a plurality of wavelength ranges, which may be derived from one or more light sources 40. Preferably, the wavelength adapting device 10 of the present invention may include a monochromatic source light 42, but a person of skill in the art will appreciate bichromatic and polychromatic light sources 40 to be included within the scope and spirit of the present invention.

For the sake of clarity, references to a source light 42, and its corresponding level of light emitted within the biological affective wavelength range, should be understood to include the light emitted by the one or more light sources 40. Correspondingly, a source light that includes a high level of light within a biological affective wavelength range should be understood to be inclusive of the wavelength ranges included in monochromatic, bichromatic, and polychromatic source lights 42.

The wavelength conversion material 30 will now be discussed in greater detail. The wavelength conversion material 30 may include in the bulk of another material, such as an optic, or be applied to another device as a conversion coating. The wavelength conversion material 30 may alter the level of light within the biological affective wavelength range of the source light transmitted from the light source 40 into a converted light with a different level of biological affective wavelength range.

As will be appreciated by a person of skill in the art, the wavelength conversion material 30 may be positioned in virtually any location where it may receive source light 42 to be converted into a converted light 46. For example, the wavelength conversion material 30 may be located between the light source 40 and a desired output direction 60. In this configuration, the wavelength conversion material 30 may convert the source light 42 into the converted light 46 prior to directing the converted light 46 in the desired output direction 60. Additionally, for example, the wavelength conversion material 30 may also be located adjacent to the light source 40. Further, for example, the wavelength conversion material 30 may be located in line with, or adjacent to, one or more mirror 33 that may reflect light that has been converted, or will be converted, in the desired output direction 60.

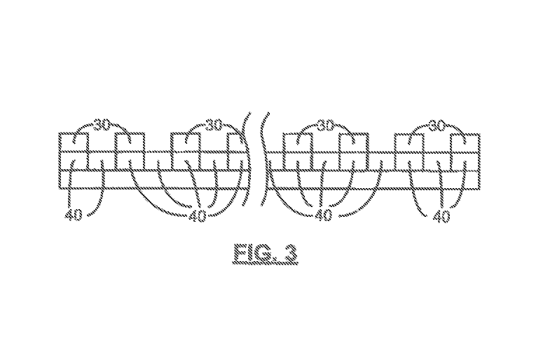

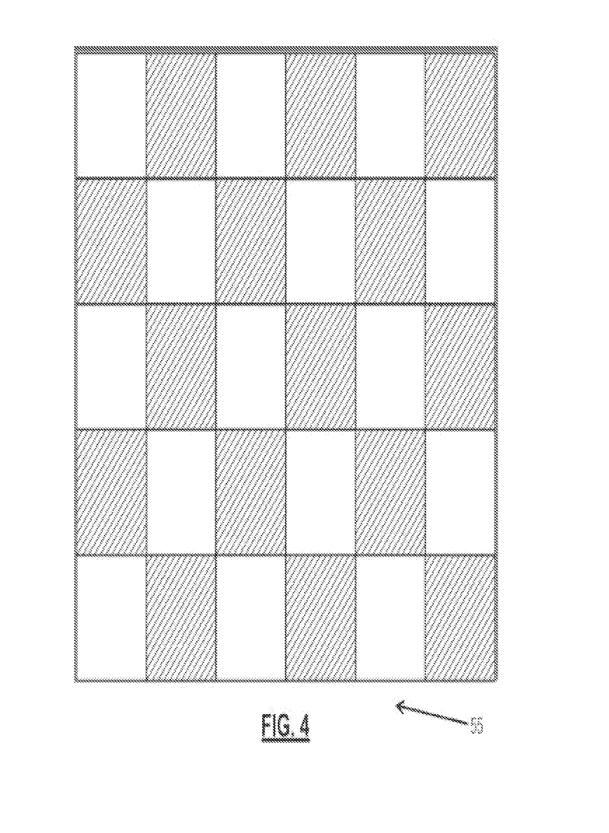

As perhaps best illustrated in FIGS. 3-4, the wavelength adapting device 10, according to an embodiment of the present invention, may include an array 55 of light sources 40. As previously discussed, the light sources 40 may be, for example, LEDs. Although the following example contemplates the inclusion of LEDs as the light sources 40 to be included within the array 55, a person of skill in the art will appreciate that any additional light source 40 that may emit a light would be included within the scope of the present invention. FIG. 3 illustrates a cross sectional view of a partial row light sources 40. The light sources 40 included in the array 55 may be sequentially aligned, allowing the array 55 of light sources to evenly emit light. The light sources included in the array 55 may be connected to a controller 61 to control the operation of individual or collective groups of light sources 40 within the array 55. The controller 61 has been described in greater detail above. A person of skill in the art will appreciate that although the light sources 40 have been illustrated with a sequential arrangement herein, virtually any arrangement may be used to produce the emission of light with a desired distribution of luminance to be included within the scope of the present invention.

As illustrated in FIG. 3, with additional reference to FIG. 4, a wavelength conversion material 30 may be located adjacent to one or more light sources 40 included within the array 55. Light sources 40 with an adjacently located wavelength conversion material 30 may be referred to as altered light sources 52. Conversely, light sources 40 without an adjacently located wavelength conversion material 30 may be referred to as normal light sources 53. The light sources 40 in the array 55 may be configured in a varying pattern of normal light sources 53 and altered light sources 52. The examples illustrated in FIGS. 3-4 illustrate the arrangement of altered light sources 52 and normal light sources 53 with a regular alternating interval, forming a pattern that may resemble a checker-board. However, a person of skill in the art will appreciate virtually any configuration of altered light sources 52 and normal light sources 53 that may allow the emission of light from one or more of the included light sources 40 to be included within the scope of the present invention.

Additionally, the altered mode may include multiple subset modes, such as an increasing mode and a decreasing mode. The increasing mode may increase the level of light in the biological affective wavelength range to be included in a converted light 46. Conversely, the decreasing mode may decrease the level of light in the biological affective wavelength range to be included in the converted light 46. By altering the level of light within the biological affective range included in the converted light 46, the wavelength adapting device 10 of the present invention may affect a corresponding physiological response. A person of skill in the art will appreciate that any number additional modes, including a continuously variable range of modes between the increasing mode, normal mode, and decreasing mode, may be included to corresponding with different desired levels of light within the biological affective range to be included in the converted light 46.

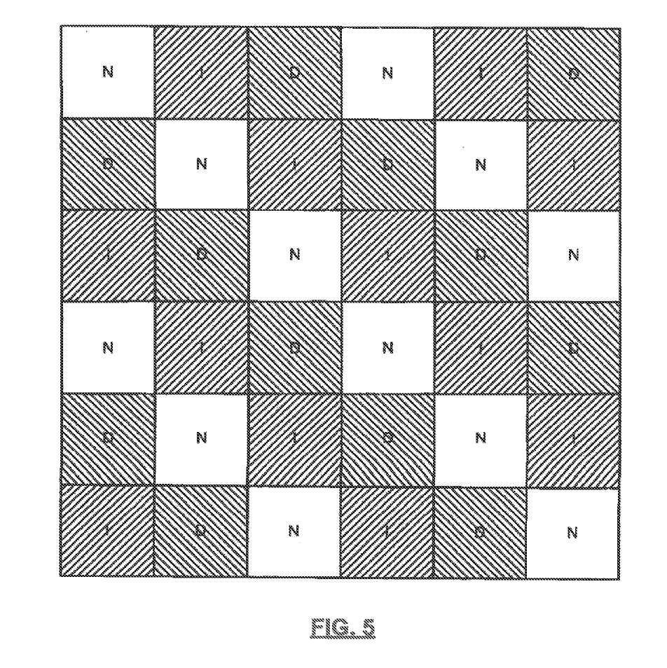

As illustrated in FIG. 5, a plurality of wavelength conversion materials 30 may be located adjacent to one or more light sources 40 included within the array 55. Similar to the array 55 of light sources 40 illustrated in FIGS. 3-4, the array 55 of FIG. 5 may include normal light sources 53 and altered light sources 52. However, the altered light sources may additionally be referred to as increasing light sources 521 and decreasing light sources 52D, which may increase or decrease the level of affective light to be included in the converted light 46 within the biological affective wavelength range, respectively. The light sources 40 in the array 55 may be also be configured in a varying pattern of normal light sources 53, increasing light sources 521, and decreasing light sources 52D. The example illustrated in FIG. 5 illustrates the arrangement of light sources 40 with a regular alternating interval. However, a person of skill in the art will appreciate virtually any configuration of altered light sources 52 and normal light sources 53 that may allow the emission of light from one or more of the included light source 40. Additionally, a person of skill in the art will appreciate the inclusion of additional light sources 40 with adjacently located wavelength conversion materials 30, which may perform additional wavelength conversions of the source light 42 into the converted light 46 resulting in a different level of lighting within the biological affective wavelength range, to be included within the scope of the present invention.

The wavelength conversion material 30 may also be movably positioned between a plurality of positions, for example, as included in or applied to an movable optic, such as an engaged position to convert source light 42 into converted light and a disengaged position wherein the source light is not converted. A person of skill in the art will appreciate that positioning of the wavelength conversion material 30 between the aforementioned positions may include the engaged position to allow operation in an altered mode, the disengaged position to allow operation in a normal mode, or any intermediate position ranging between the engaged position and the disengaged position.

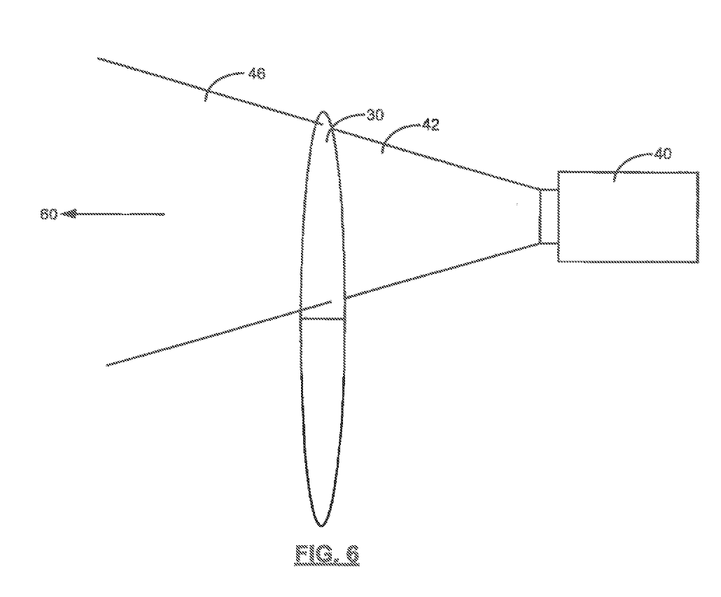

As perhaps best illustrated in FIG. 6, the moveable positioning of the wavelength conversion material 30 may occur through rotation. A person of skill in the art will appreciate the rotatable positioning to include rotating the wavelength conversion material 30 in the clockwise and/or counterclockwise direction. Wavelength conversion materials 30 that may be rotatably positioned may also include an electromechanical device to provide physical motion. The wavelength conversion material 30 may be located adjacent to a rotatable disc, such as a color wheel. A person of skill in the art will appreciate that a rotatable disc is given as an example, and that any member that may be rotated between an engaged position and a disengaged position should be included within the scope of the present invention. Additionally, a person of skill in the art will appreciate the inclusion of additional rotatable discs, which may perform additional wavelength conversions, such as color conversions, as within the scope of the present invention.

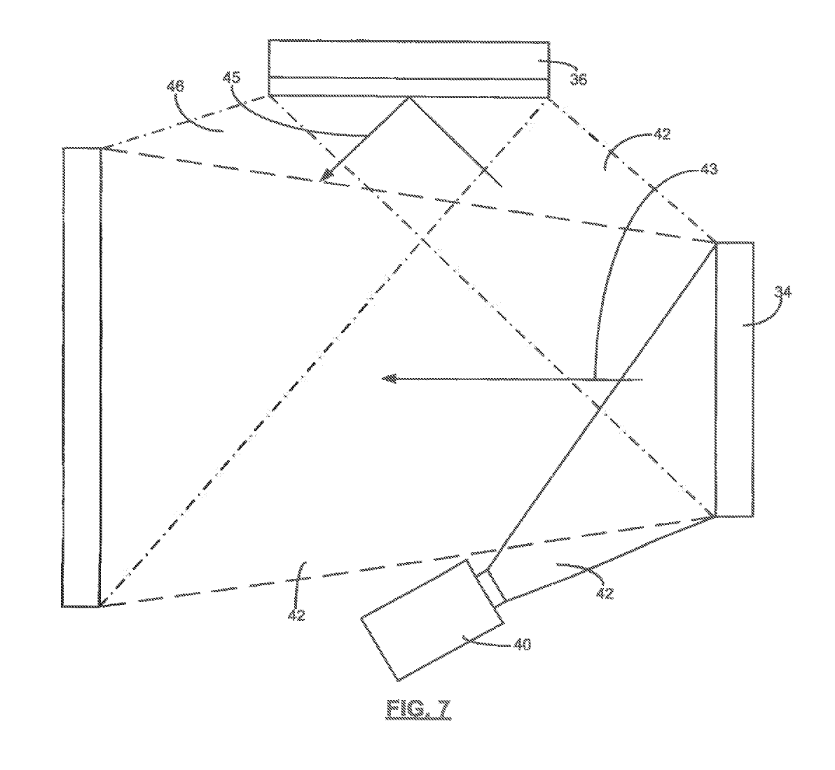

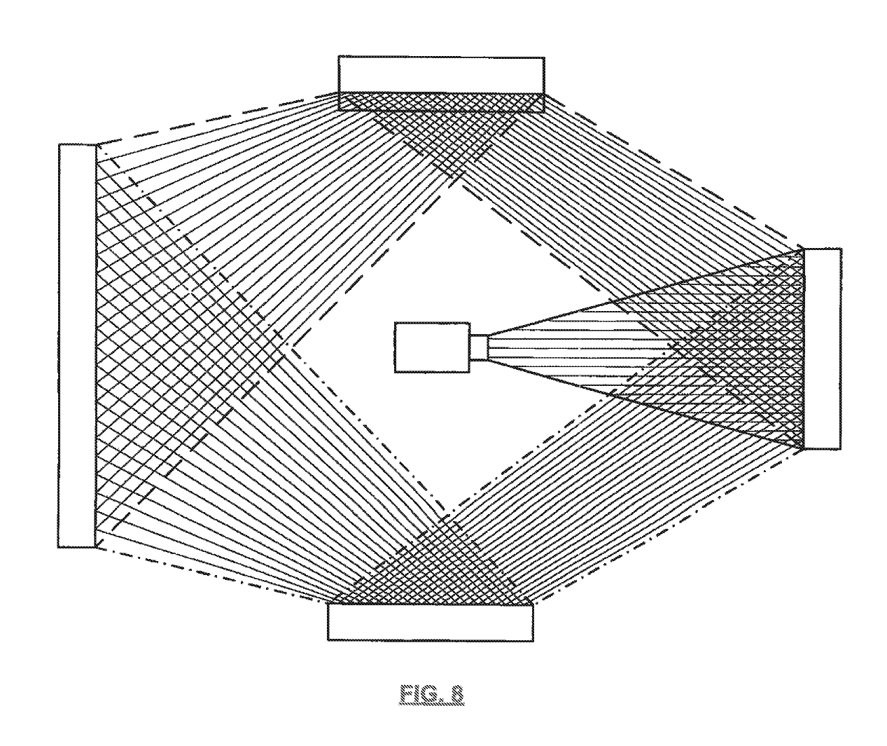

Referring additionally to FIGS. 7-8, the wavelength adapting device 10 of the present invention may include one or more mirror 33. The mirror 33 may be stationary or movable. As would be apparent to a person of skill in the art, the mirror 33 may include a reflective surface. The reflective surface may receive and reflect light. The wavelength conversion material 30 may be located adjacent to the reflective surface of the mirror, such to convert a source light 42 that may be received and reflected.

One or more mirror included in the wavelength adapting device 10 of the present invention may be a stationary mirror. The inclusion of stationary mirrors is illustrated in FIGS. 7-8. FIG. 8 further illustrates the inclusion of a first mirror 34 and a second mirror 36. A person of skill in the art will appreciate that the present example has been provided for illustrative purposes only, and should not be considered to limit locating stationary mirrors to the illustrated positions.

A wavelength conversion material 30 may be located adjacent to the reflective surface of one or more of the mirrors 33, such as the first mirror 34. Alternatively, the wavelength conversion material 30 may be located at an intermediate position between the light source 40 and the mirror 33, and/or between the mirror 33 and the desired output direction 60. A person of skill in the art will appreciate that an optic including a conversion material 40 may be located adjacent to a plurality of mirrors 33, as it would be included within the scope and spirit of the present invention.

An example of a movable mirror may include a repositionable mirror 38, which may be configurable among a plurality of positions to reflect light in a desired direction 60. A person of skill in the art will appreciate that the repositionable mirror 38 may be configured in a virtually limitless number of positions, and may be continuously varied between those positions. By varying the position of the repositionable mirror 38, the wavelength adapting device 10 of the present invention may control the quantity of light reflected in the desired direction 60. A person of skill in the art will appreciate additional operations that may control the quantity of light reflected in a desired direction, such as, but not limited to, pulse width modulation. The wavelength conversion material 30 may be located adjacent to the face of the repositionable mirror 38. However, skilled artisans will appreciate that mirrors 33 included in embodiments of the present invention need not be repositionable to be contemplated by the present invention.

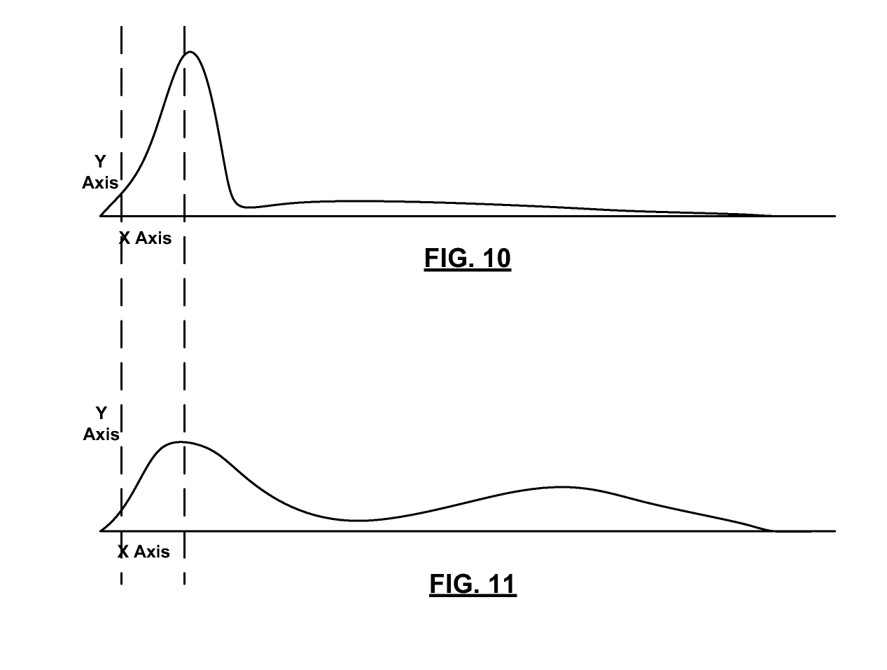

The light converting device 10, according to an embodiment of the present invention, may include a single repositionable mirror 38. The light converting device 10 may also include a plurality of repositionable mirrors 38, which may be included in an array 55 of mirrors. The wavelength conversion material 30 may be located adjacent to the reflective surface of one or more of the repositionable mirrors 55 included in the array 55. The repositionable mirrors 38 may be arranged in a configuration similar to the array 55 of light sources 40 with wavelength conversion materials 30 being located adjacent to the repositionable mirrors 38, as described above (FIGS. 10-11). The repositionable mirrors 38 included in an array 55 of mirrors may each selectively or collectively reflect light in a desired direction 38. This selective reflection may be controlled by a controller 61, the operation of which will be discussed in greater detail below.

Referring now to FIG. 7, an example of an embodiment of the wavelength adapting device 10 of the present invention including a stationary first mirror 34 and a repositionable mirror 38 will now be discussed. A wavelength conversion material 30 may be located adjacent to the first mirror 34 to convert the source light 42 received and reflected by the first mirror 34 into a converted light 46. The light source 40 may emit a source light 42 in the direction of the repositionable mirror 38. The repositionable mirror 38 may be adjusted to reflect the source light 42 directly in a desired output direction 60. The repositionable mirror 38 may also be adjusted to reflect the source light 42 in the direction of the first mirror 34 with the adjacently located conversion material 30. At the first mirror 34, the source light 42 may be converted into the converted light 46 and reflected in the desired output direction 60.

Referring additionally to FIG. 8, an example of an embodiment of the wavelength adapting device 10 of the present invention including a stationary first mirror 34, a stationary second mirror 35, and a repositionable mirror 38 will now be discussed. A wavelength conversion material 30 may be located adjacent to the first mirror 34 to convert the source light 42 received and reflected by the first mirror 34 into a converted light 46. The light source 40 may emit a source light 42 in the direction of the repositionable mirror 38. The repositionable mirror 38 may be adjusted to reflect the source light 42 in the direction of the second mirror 35, which may subsequently reflect the source light 42 in a desired output direction 60 without the performance of a wavelength conversion. The repositionable mirror 38 may also be adjusted to reflect the source light 42 in the direction of the first mirror 34. At the first mirror 34, the source light 40 may be converted into the converted light 46 and reflected in the desired output direction 60.

The repositionable mirrors 38 included in an array 55 of mirrors may be micromirrors. The micromirrors may be included in a microelectromechanical system (MEMS). A MEMS device, with selective application of wavelength conversion coatings, may be further described in U.S. patent application Ser. No. 13/073,805 to Maxik, et al., the entire contents of which is incorporated herein by reference.

In an embodiment wherein the wavelength adapting device 10 includes a wavelength conversion coated MEMS device, the adjacent location of wavelength conversion materials 30 to the individual micromirrors may resemble the pattern illustrated in FIGS. 4-5, relating to a pattern of wavelength conversion materials 30 being located adjacent to a plurality of light sources 40. Additionally, source light 42 may be reflected by a MEMS device, or virtually any array 55 of repositionable mirrors 38, such that it may pass through a wavelength conversion material 30 located elsewhere than adjacent to the reflective surface of the repositionable mirror 38.

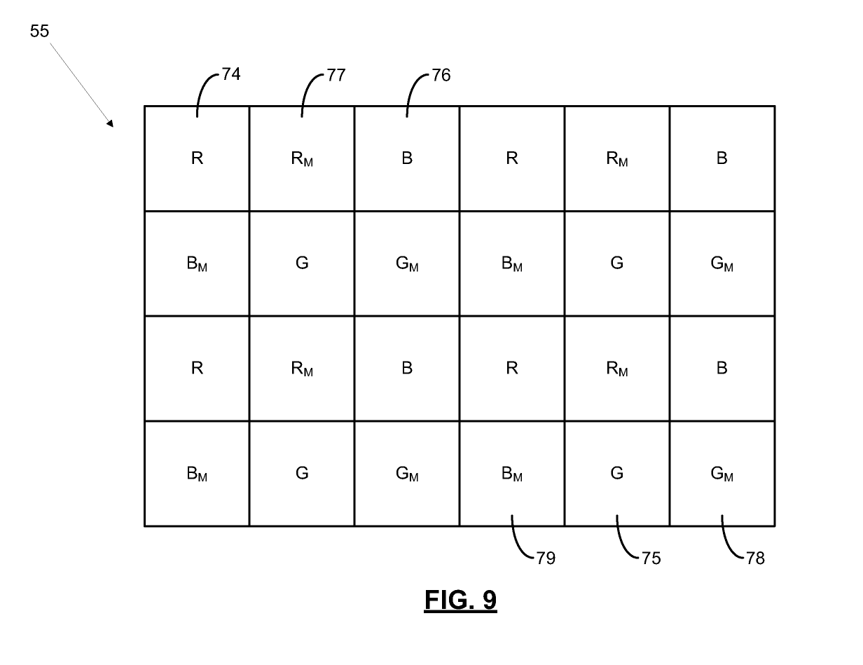

Additionally, as perhaps best illustrated in FIG. 9, the wavelength adapting device 10 of the present invention may include a plurality of wavelength conversion materials 30 configured to perform a conversion of light within the biological affective wavelength range and a color conversion. By providing performing color conversion, and alteration of the level of light within the biological affective wavelength range, the wavelength adapting device 10 of the present invention may advantageously integrate multiple wavelength conversion operations into one device. This integration may beneficially reduce required number of parts and overall complexity of a device in which the wavelength adapting device 10 may be implemented.

For clarity, the array 55 of wavelength conversion materials 30 illustrated in FIG. 9 may provide a color conversion from a source light 42 into a converted light 46 within the red, green, and blue color wavelength ranges with a first level of biological affective light (74, 75, and 76, respectively). The array illustrated in FIG. 9 may also provide a color conversion from a source light 42 into a converted light 46 with red, green, and blue color wavelength ranges with a second level of biological affective light (77, 78, and 79, respectively). The array 55 of wavelength conversion materials 30 illustrated in FIG. 9 may provide for each color conversion to be performed with or without a wavelength conversion that alters the level of light within the biological affective wavelength range.

For example, a video signal may define a violet colored light with increased melatonin production. In an embodiment with selectively enabled LEDs located adjacent to wavelength conversion materials 30, the wavelength adapting device 10 may then control the LEDs located adjacent to the red-melatonin wavelength conversion material 77 and blue-melatonin wavelength conversion material 79 to emit light, creating the desired light within the indicated wavelength range. A person of skill in the art will appreciate additional combinations of the wavelength conversion materials 30 to produce a desired effect. These combinations may include the emission of light by one or more LED located adjacent to a conversion material 30 that may alter the biological affective wavelength range and one or more LED located adjacent to a conversion material 30 that may not alter the biological affective wavelength range.

A person of skill in the art will additionally appreciate that the wavelength adapting device 10 of the present invention may include a plurality of wavelength conversion materials 30. The plurality of wavelength conversion materials 30 may be positioned, collective or separately, adjacent to the light source 40, mirror 33, desired output direction 60, and/or at an intermediate position between the one of the aforementioned locations.

In this disclosure, the wavelength conversion material 30 may be included in an optic as a structural element to be located between the light source 40 and the desired output direction 60. The wavelength conversion material 30 may additionally be located adjacent to, or in line between, a mirror 33 that may receive source light 42 from the light source 40, as perhaps best illustrated in FIGS. 7-8. In this embodiment, the mirror may be located adjacent to a substantially stationary wavelength conversion material 30. However a person of skill in the art will appreciate embodiments wherein the adjacent wavelength conversion material 30 may be movable. Source light 42 may pass through the wavelength conversion material 30 prior to being received by the mirror 33. Similarly, source 42 may pass through the wavelength conversion material 30 after being reflected by the mirror 33, as it may be projected in the desired output direction 60 as converted light 46.

As discussed above, the wavelength conversion material 30 may be movably or rotatably positioned to be located adjacent to the light source 40 and/or a mirror 33. The wavelength conversion material 30 may be connected to an electromechanical device, which may orient the wavelength conversion material 30 between the engaged position and the disengaged position. Electromechanical devices may include, but should not be limited to, motors, pistons, actuators, electromagnetic devices, pneumatics, hydraulics, and other devices capable of generating motion.

To provide the wavelength conversion operation, the wavelength conversion material 30 may absorb light within a first wavelength range and emit light within a second wavelength range, as will be discussed in greater detail below. By altering wavelength ranges through absorption and emission, and not through filtration and blocking of selected wavelength ranges, the energy of the light is not substantially lost. Filtration and light blocking techniques may remove a wavelength range of a source light 42, resulting in light energy being lost from the removed light. Additionally, this lost light energy may be converted into heat energy, which may further diminish the efficiency of an adjacently located light source 40, such as an LED. The conservation of energy provided by the embodiments of the present invention may advantageously allow for enhanced operational efficiency during operation.

The wavelength conversion material 30 may alter the first level of affective light in the biological affective wavelength range included in the source light 42 into a second level of affective light included in the converted light 46 will now be discussed in greater detail. An example of a source light 42, which may include a first level of affective light, is illustrated as the waveform 84 of FIG. 10. The conversion materials 30 are preferably provided by a fluorescent or phosphorescent material, such as a phosphor and/or quantum dot, capable of converting a light with a source wavelength range into a light with one or more converted wavelength ranges.

More specifically, the wide production conversion material may include a phosphorous wavelength conversion material. Also, the narrow production conversion material may include a quantum dot wavelength conversion material. However, it will be appreciated by skilled artisans that any conversion material 30 that may be capable of converting a light from one wavelength range to another wavelength range may be included in the wavelength conversion material 30 and be included within the scope and spirit of the present invention.

A wide production conversion material, such as a material based on a phosphorous material, may alter the wavelength range of light absorbed by the material. A source wavelength range may be converted into one or more converted wavelength ranges that may include levels light in the biological affective wavelength range that differ from the source light 42.

A phosphor substance may be illuminated when it is energized. Energizing of the phosphor may occur upon exposure to light, such as the source light 42 emitted from the light source 40. The wavelength of light emitted by a phosphor may be dependent on the materials from which the phosphor is comprised. Typically, phosphors may convert a source light 42 into a converted light 46 within a wide converted wavelength range, as will be understood by skilled artisans.

Additionally, a narrow production conversion material, such as a material based on quantum dots may alter the wavelength range of light absorbed by the material. A source wavelength range may be converted into one or more converted wavelength ranges. Similarly to the wavelength conversion performed by the wide production conversion material, the converted wavelength ranges may include levels of light in the biological affective range that differ from the source light 42.

A quantum dot substance may also be illuminated when it is energized. Energizing of the quantum dot may occur upon exposure to light, such as the source light 42 emitted from the light source 40. Similar to a phosphor, the wavelength of light emitted by a quantum dot may be dependent on the materials from which the quantum dot is comprised. Typically, quantum dots may convert a source light 42 into a converted light 46 within a narrow converted wavelength range, as will be understood by skilled artisans.

The conversion of a source wavelength range into a converted wavelength range may include a shift of wavelength ranges, which may be known to those skilled in the art as a Stokes shift. During a Stokes shift, a portion of the source wavelength range may be absorbed by a conversion material 30. The absorbed portion of source light 42 may include light within the biologically affective wavelength range. This absorption may result in a decreased intensity of light within the source wavelength range.

The portion of the source wavelength range absorbed by the conversion material may include energy, causing the atoms or molecules of the conversion coating to enter an excited state. The excited atoms or molecules may release some of the energy caused by the excited state as light. The light emitted by the conversion coating may be defined by a lower energy state than the source light 42, which may have caused the excited state. The lower energy state may result in wavelength ranges of the converted light 46 defined by light with longer wavelengths. A person of skill in the art will appreciate additional wavelength conversions that may emit a light with shorter wavelength ranges to be included within the scope of the present invention, as may be defined via the anti-Stokes shift.

As will be understood by a person of skill in the art, the energy of the light absorbed by the wavelength conversion material 30, which may include a conversion coating, may shift to an alternate energy of light emitted from the wavelength conversion material 30. Correspondingly, the wavelength range of the light absorbed by the conversion coating may be scattered to an alternate wavelength range of light emitted from the conversion coating. If a light absorbed by the conversion coating undergoes significant scattering, the corresponding emitted light may be a low energy light within a wide wavelength range. Substantial scattering characteristics may be definitive of a wide production conversion coating. Conversely, if the light absorbed by the conversion coating undergoes minimal scattering, the corresponding emitted light may be a high energy light within a narrow wavelength range. Minimal scattering characteristics may be definitive of a narrow production conversion coating.

Color conversion coatings may be used to convert the source light emitted by a light source, such as, for example, a blue light LED, into a more desirable white converted light. An example of a color converted light is illustrated as waveform 85 of FIG. 11. However, as seen in waveform 85, the wavelength conversion performed by the a color conversion coating may result in a significant portion of the affective light in the biological affective wavelength range remaining unconverted from the source light 42. To convert a substantial portion of the remaining high level of affective light into a converted light 46 with a low level of affective light, a wavelength conversion operation may advantageously be performed by the wavelength adapting device 10 of the present invention to increase or reduce the level of affective light included in the converted light.

As source light 42 is emitted from a light source 40, it may include a narrow wavelength range of high energy light. This high energy light may be within the biological affective wavelength range, as perhaps best illustrated by the wavelength range 84 of FIG. 10, without limitation. As the wavelength conversion material 30 may convert the source light 42 into a converted light, the wavelength conversion material 30 including wide production conversion coating materials may convert a portion of the biological affective wavelength range of high energy light in the into a wide wavelength range of low energy light. This low energy light may include, for example and without limitation, yellow, orange, and red light. This low energy light may additionally omit high levels of affective light in the biological affective wavelength range.

Additionally, a wavelength conversion material 30 including narrow production conversion coating materials may convert a portion of the affective light in the biological affective wavelength range into one or more alternate narrow wavelength range of low energy light. The converted light produced by the narrow production conversion coating may include one or more narrow wavelength range of high energy and/or low energy light. This alternate narrow wavelength range may include levels of affective light that are different than the levels of affective light included in the source light.

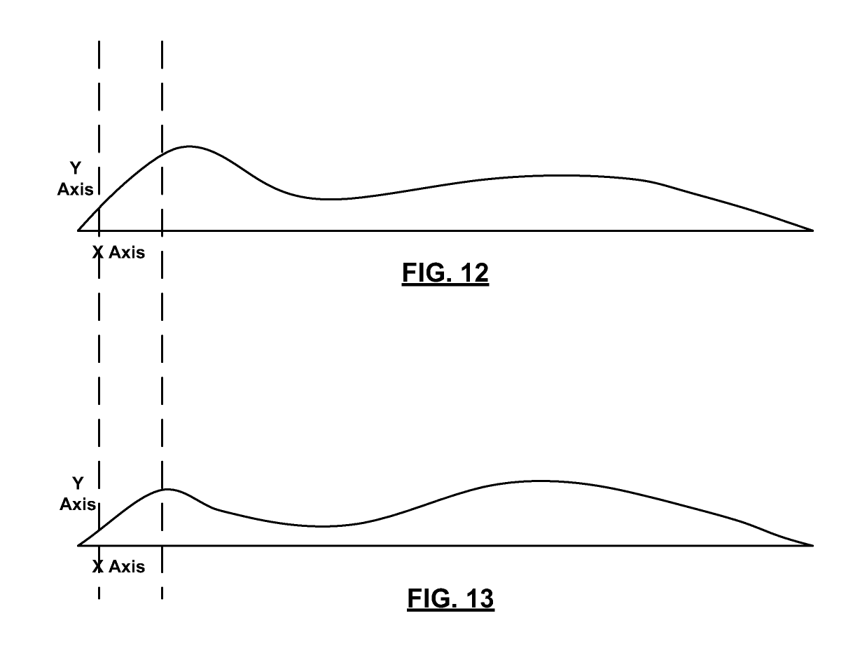

The wavelength conversion material 30 may operate in an increasing mode or a decreasing mode, respectively increasing or decreasing the level of affective light to be included in the converted light. In the increasing mode, the inclusion of wide production conversion coating materials in the wavelength conversion material 30 may allow the displacement of light from outside of the biological affective wavelength range to a broad high energy wavelength range, which may include light within the biological affective wavelength range, as perhaps best illustrated, for example, by waveform 86 of FIG. 12. Alternatively, in the decreasing mode, the inclusion of wide production conversion coating materials in the wavelength conversion material 30 may allow the displacement of light from the biological affective wavelength range to a broad low energy wavelength range, as illustrated, for example, by waveform 87 of FIG. 13.

Additionally, the inclusion of narrow production conversion coating materials in the wavelength conversion material 30 may allow the wavelength adapting device 10 to selectively displace wavelength ranges included in the source light. These wavelength ranges may include, for example and without limitation, the biological affective wavelength range. In the increasing mode, the inclusion of narrow production conversion coating materials in the wavelength conversion material 30 may allow the displacement of light from outside of the biological affective wavelength range to a narrow high energy wavelength range, which may include light within the biological affective wavelength range. This narrow production wavelength conversion performed in the increasing mode may perhaps be best illustrated, for example, by waveform 88 of FIG. 14.

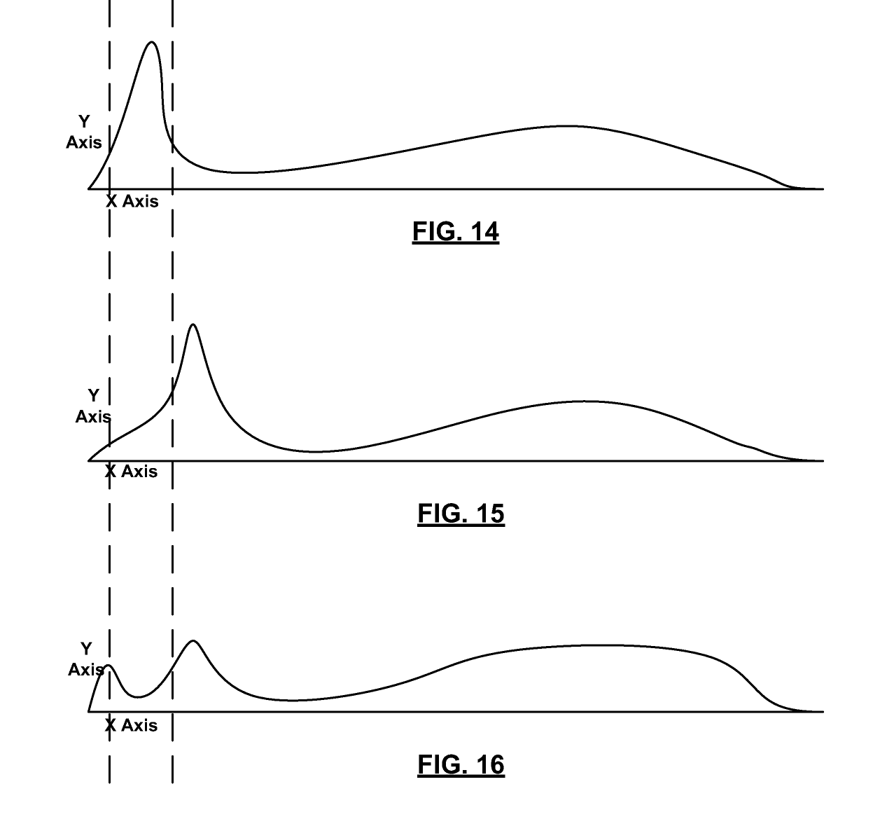

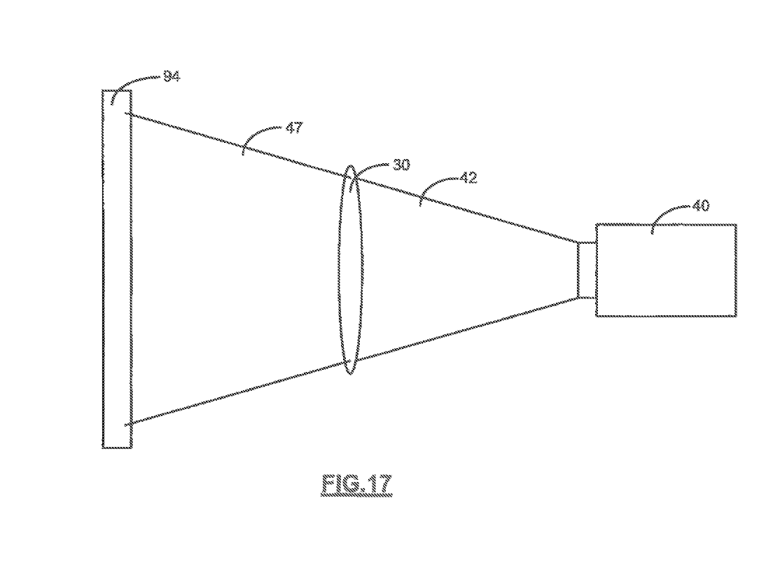

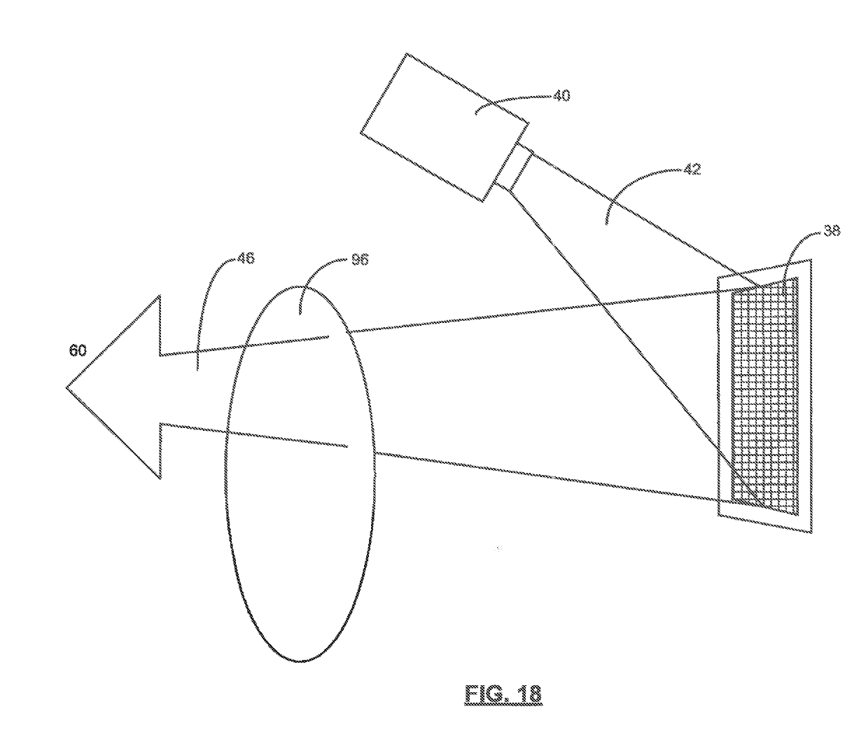

In the decreasing mode, the inclusion of narrow production conversion coating materials in the wavelength conversion material 30 may allow the displacement of light from the biological affective wavelength range to one or more alternate narrow wavelength range. The displacement may be illustrated, for example, by waveforms 89,90 of FIGS. 15 and 16. Referring to FIGS. 10 and 15, the narrow production conversion coating may receive a source light, which may be illustrated by waveform 84. The narrow production conversion coating may then displace a level of the affective light, which was included in the biological affective wavelength range, via a Stokes shift absorption and emission. The converted light, including the light displaced form the biological affective wavelength range, may be illustrated by waveform 89. Here, the wavelength conversion material 30 with the narrow production conversion coating may produce a converted light with high level of light within a narrow, high energy wavelength range that may approximate, but not equal, the biological affective wavelength range. By approximating, but not equaling, the biological affective wavelength range, the wavelength adapting device 10 of the present invention may produce a converted light with an altered physiological response, but essentially the same chromaticity, as the source light.

Referring to FIGS. 10 and 16, an alternate example of a wavelength conversion material 30, including a narrow production conversion coating and operating in the decreasing mode, will now be discussed. One or more narrow production conversion coatings may receive a source light, which may be illustrated by waveform 84. The narrow production conversion coatings may then displace a level of the affective light, which was included in the biological affective wavelength range, via a Stokes shift absorption and emission. The converted light, including the light displaced form the biological affective wavelength range, may be illustrated by waveform 90. Here, the wavelength conversion material 30 with the narrow production conversion coating may produce a converted light with high level of light within a plurality of narrow wavelength ranges, which may combine or average to collectively approximate, but not equal, the biological affective wavelength range. By approximating, but not equaling, the biological affective wavelength range, the wavelength adapting device 10 of the present invention may produce a converted light with an altered physiological response, but essentially the same chromaticity, as the source light.

A person of skill in the art will appreciate embodiments of the wavelength conversion material 30 that may include a plurality of wide production and narrow production conversion coatings to be included within the scope of the present invention. In an example wherein the source light 42 includes a wide production and narrow production conversion coating to absorb and convert a biological affective wavelength range of high energy light, the wide production conversion coating may convert a portion of the blue light into a wide wavelength range of light defined by longer wavelengths, such as yellow, orange, and red light. The light produced by the wide production conversion coating may be supplemented with a wavelength conversion performed by the narrow production conversion coating. By including a combination of wide production and narrow production conversion coatings, the wavelength conversion material 30 may advantageously, and more efficiently, convert a source light including a first level of affective light into a converted light including a second level of affective light.

While operating in the decreasing mode, although the level of affective light included in the converted light may be substantially reduced from the source light, at least part of affective light may remain after the wavelength conversion operations have been performed. However, it will be appreciated by skilled artisans that the substantial reduction of affective light within the converted light may produce essentially the same physiological response as if the converted light was absent any affective light.

The aforementioned example has been included to describe a wavelength conversion operation to reduce the level of light within the biological affective wavelength range, according to the Stokes shift. A person of skill in the art will appreciate that an operation, converse the above described example, may be performed in accordance to the anti-Stokes shift. Skilled artisans will further appreciate that the converse operation may be performed to increase the level of light in the biological affective wavelength range to be included in the converted light.

As will be additionally understood by those skilled in the art, the source light 42 within a source wavelength range may be converted by a wavelength conversion material 30 that may include a wide production conversion coating into a converted light with multiple wavelength ranges. The use of multiple wide production conversion coatings, such as phosphors, may produce a light that includes multiple discrete and/or overlapping wavelength ranges. These wavelength ranges may be combined to produce the converted light. A person of skill in the art will appreciate that references to a converted within this disclosure, and its corresponding wavelength ranges, should be understood to include all wavelength ranges that may have been produced as the source light 42 may pass through the wide production conversion coating 30, which may be included in the wavelength conversion material 30.

Similarly, the source light within the source wavelength range may be converted by a wavelength conversion material 30 including a narrow production conversion coating into a converted light 46 with multiple converted wavelength ranges. The use of multiple narrow production conversion coatings, such as quantum dots, may produce a light that includes multiple discrete and/or overlapping wavelength ranges. These wavelength ranges may be combined to produce the converted light 46. A person of skill in the art will appreciate that references to a converted light 46, and its corresponding converted wavelength ranges, should be understood to include all wavelength ranges that may have been produced as the source light may pass through the narrow production conversion coating, which may be included in the wavelength conversion material 30.

A person of skill in the art will appreciate that one or more additional wavelength conversion material 30s may be included in the wavelength adapting device 10 of the present invention. The additional wavelength conversion material 30s may be used to perform additional, or supplemental, conversion coatings to increase or decrease the level of light within the biological affective wavelength range to be included in the converted light. Alternatively, an additional conversion coating may be included to perform a wavelength conversion to alter the chromaticity of the converted light such to appear as a desired output color.

The inclusion of one or more wavelength conversion material 30s intended to convert the color of the light may alter the wavelength range of the source light into a desired wavelength range of the converted light as described above. By absorbing source light within one wavelength range and converted light in a different wavelength range, the color wavelength conversion coating may similarly perform a Stoke shift or an anti-Stokes shift.

By including a combination of wavelength conversion material 30s, the wavelength adapting device 10 of the present invention may alter the level of light in the biological affective wavelength range without significantly changing the chromaticity of the converted light, resulting in a substantially similar perceived output color between the source light and the converted light. Alternatively, by including a combination of wavelength conversion coatings within a wavelength conversion material 30, the wavelength adapting device 10 of the present invention may similarly alter the level of light in the biological affective wavelength range without significantly changing the chromaticity of the converted light. Ultimately, the converted light created by the wavelength conversion operation may be nearly indistinguishable between the source light with a first level within the biological affective wavelength range and the converted light with a second level within the biological affective wavelength range.

The physiological response, which may be affected by a stimulus such as light within the biological affective wavelength range, will now be discussed. A person of skill in the art will appreciate that physiological responses may occur in virtually any living organism. In the interest in clarity, the following discussion may be limited to physiological responses as they may relate to humans. However, a skilled artisan will understand that such limitation in the following discussion is not intended to limit the scope of the present invention to the physiological responses experienced by humans, and would appreciate the broad application of the following discussion to apply to all living organisms.

At the basic level of biology, substantially all multicellular organisms produce and receive hormones. Cellular functions may be manipulated and controlled through the instructional messages transmitted throughout the body through the manufacture and distribution via hormones. This manufacture and distribution of hormones may be defined as a physiological response. Cells may include receptors tailored to sense and receive one or more hormones. Upon the sensation of a hormone by the receptor, the cell may alter its behavior. This alteration of cellular behavior may be defined as a biological effect.

In the interest of clarity, a biological effect discussed below may describe an adjustment of a circadian rhythm cycle experienced by an organism. Additionally, the physiological response discussed below may apply to melatonin production, which may affect the circadian rhythm cycle of an organism. A person of skill in the art will appreciate that the discussion of a specific biological effect and physiological response is included herein in the interest of clarity, and that a plurality of biological effects and physiological responses are intended to be included within the scope of the present invention.

A circadian rhythm may be best defined by looking to the root of the term. “Rhythm” may be defined as a fluctuation or variation marked by regular recurrence or natural flow. “Circadian” may be defined as being characterized by an approximately twenty-four hour cycle. Thus, a circadian rhythm may naturally be defined as a regular fluctuation of a biological effect within an approximately twenty-four hour cycle. One such biological effect subject to a circadian rhythm may include, for example, the onset of sleepiness.

As will be known by a person of skill in the art, the circadian rhythm may informally be referred to as a “biological clock.” However, like the hands of a clock, a circadian rhythm may be adjusted. The natural circadian rhythm of a human, absent all external stimulus, may approximate, but not equal, a twenty-four hour cycle. However, the effective circadian rhythm may be approximately synchronized with the twenty-four hour cycle of the earth through the use of zeitgebers. A zeitgeber may be defined as an external cue received by an organism that is used to synchronize its internal circadian rhythm. Zeitgebers that may affect a circadian rhythm may include temperature, exercise, eating and drinking habits, pharmaceuticals, and other external cues. However, the most influential zeitgeber is light, and, more specifically, light within the biologically affective wavelength range of 460 nanometers to 490 nanometers.

Upon the sensation of a zeitgeber, an organism may begin producing a hormone as a physiological response. For example, at approximately sunset, an organism may cease to receive the zeitgeber of sunlight including light within the biologically affective wavelength range. As a result, the body may begin manufacturing and distributing the melatonin hormone. As the melatonin is distributed throughout the body, it may be received by receptors in the brain. Upon receipt of the melatonin, the receptors may induce the onset of sleepiness as a biological effect of the hormone.

However, within a given day, a person may encounter a variety of conditions that may affect the sensation of a zeitgeber. For example, a person that spends an entire day indoors may not be exposed to natural sunlight, and thus may not receive the zeitgeber of light within the biological affective wavelength range. As a result, the body may begin producing melatonin as a response, inducing sleepiness during the day. Conversely, for example, a person may enjoy an evening television show on a display including a backlight that emits light within the biological affective wavelength range. One such backlight may include, for example, a LED backlight. As a result, the body may cease production of melatonin as a physiological response, inhibiting sleepiness at night. A person of skill in the art will appreciate that the preceding example has been included for illustrative purposes, and should not interpret any limitation into their inclusion herein.

The desired output direction 60 of the converted light 46 generated by the wavelength adapting device 10 of the present invention will now be discussed. After a source light 42 has been converted by the wavelength adapting device 10 of the present invention into a converted light 46, it may be projected in a desired output direction 60, as may perhaps best be illustrated in FIG. 1. The converted light 46 generally diffuse into a volume, such as a room.

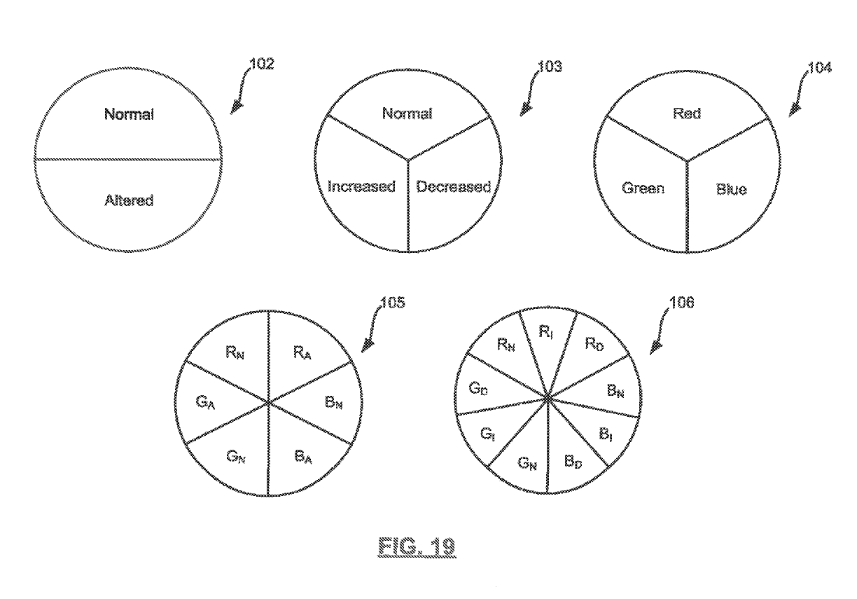

Alternatively, the converted light 46 may be received by a screen 94, as perhaps best illustrated in FIG. 17. The screen may be included in a display. The converted 46 light may also be projected to a screen, which may be included as a backlight in a display. The converted light 46 projected by the wavelength adapting device 10, which may be included as a backlight to of a display, may thus illuminate the screen.

Furthermore, the light converting device 10 of the present invention may project the converted light to a screen used to display an image. The image may be generated, for example, via plurality of micromirrors included in a MEMS device. The light reflected by the micromirrors may be passed through a rotatable disc, such as a color wheel, that may filter or convert the color of light passed through the rotatable disc.

In the following examples, additional specific embodiments of the wavelength adapting device 10 of the present invention will be discussed. A person of skill in the art will appreciate additional embodiments, which although not disclosed specifically below, would be included within the scope and spirit of the present invention. As a result, a skilled artisan should view the wavelength adapting device 10 of the present invention as limited to the examples provided below.

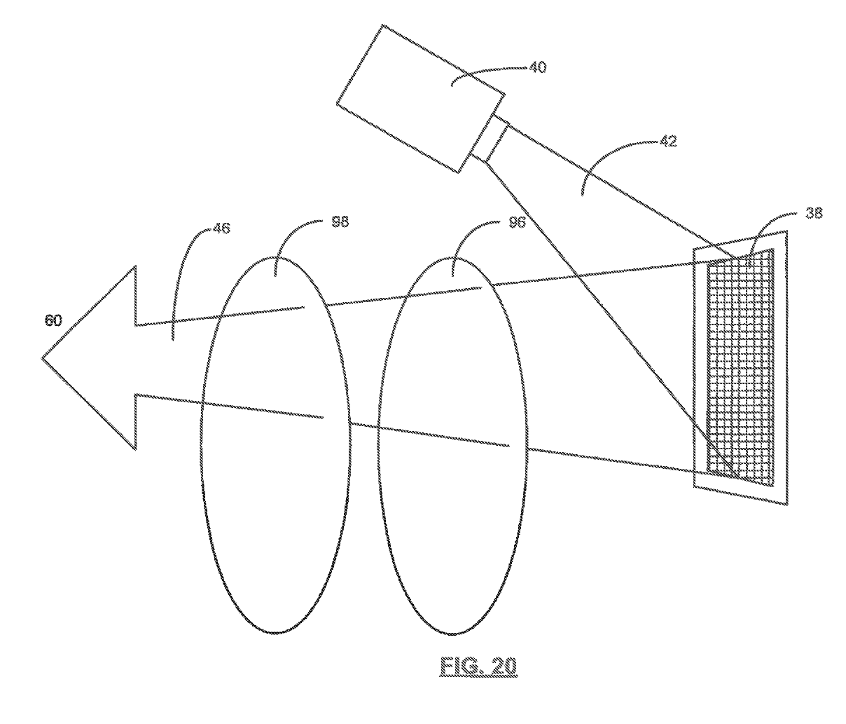

Referring now to FIG. 18, an illustrative configuration of the wavelength adapting device 10 of the present invention will now be discussed. In this example, the source light may be received by a repositionable mirror from a light source. The source light may be reflected by the repositionable mirror in the direction of a rotatable first disc. The first disc may include a wavelength conversion material 30 located adjacent to at least a part of the disc. Upon receiving the source light from the repositionable mirror, the wavelength conversion material 30 may convert the source light into the converted light.

The repositionable mirror may additionally be operatively connected to the controller 61, which may control at which intervals the source light is reflected in the direction of the first disc. The controller 61 may be synchronize the rotative position of the first disc with operation in the normal mode and the altered mode. To operate in the normal mode, the repositionable mirror may be controlled to reflect light in the direction of the first disc only when the light would not be received by the wavelength conversion material 30 located adjacent to at least part of the first disc. Conversely, to operate in the altered mode, the repositionable mirror may be controlled to reflect light in the direction of the first disc only when the light would be received by the wavelength conversion material 30 located adjacent to at least part of the first disc.

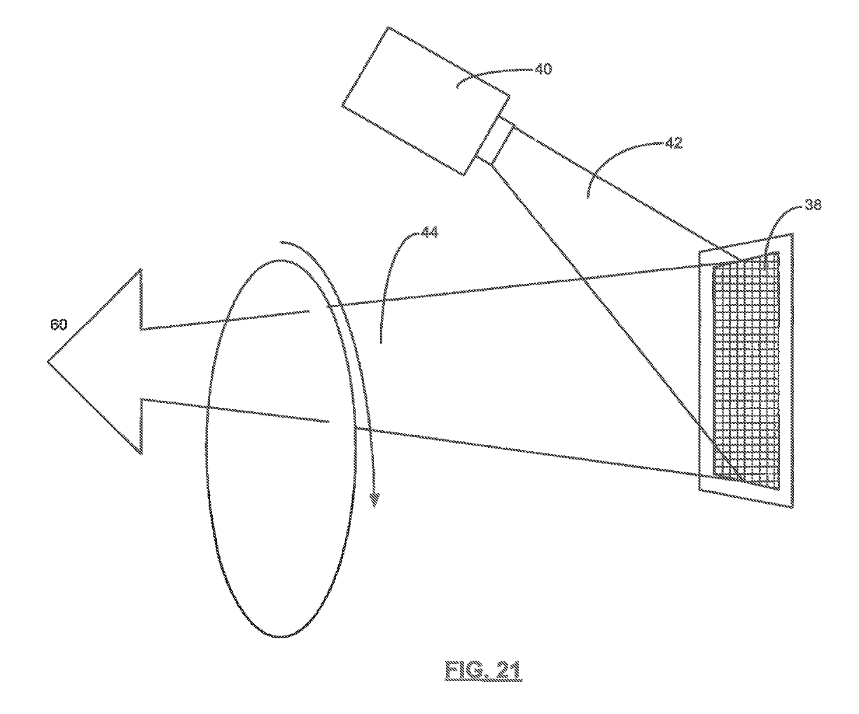

Referring now additionally to FIG. 19, the first rotatable disc 19A may include one or more adjacently located wavelength conversion material 30s. As would be understood by a person of skill in the art, the model discs 102-106 illustrated in FIG. 19 have been provided for illustrative purposes, and should not be viewed as limiting the present invention these specific examples. Model disc 102 illustrates a rotatable disc with a wavelength conversion material 30 located adjacent to approximately half of the rotatable disc, providing an approximately 50% duty cycle between operation in the normal mode and the altered mode.

Additionally, model disc 103 illustrates a rotatable disc with a plurality of wavelength conversion material 30s located adjacent to approximately two equal thirds of the rotatable disc. The adjacently located wavelength conversion material 30 may include a first wavelength conversion material 30 to increase the level of affective light in the converted light and a second wavelength conversion material 30 to decrease the level of affective light in the converted light, providing approximately 33% duty cycles between operation in the normal mode, increased mode and decreased mode.

Model disc 104 illustrates a rotatable disc with a plurality of wavelength conversion material 30s located adjacent to approximately three equal thirds of the rotatable disc. The adjacently located wavelength conversion material 30 may include a first wavelength conversion material 30 to convert the source light into the converted light with a red color, a second wavelength conversion material 30 to convert the source light into the converted light with a blue color, and a third wavelength conversion material 30 to convert the source light into the converted light with a green color. The model disc 104 may provide approximately 33% duty cycles between red, blue, and green color conversions.

Additionally, model disc 105 illustrates a rotatable disc with a plurality of wavelength conversion material 30s located adjacent to approximately six equal parts of the rotatable disc. Approximately half of the model disc 105 may include wavelength conversion material 30s located adjacent that include a wavelength conversion coating to alter the level of affective light in the converted light, providing an approximately 50% duty cycle between operation in the normal mode and the altered mode.

Each approximate half of the model disc 105 may additionally be segmented into three approximately equal parts, with may perform a color conversion with or without altering the level of affective light in the converted light. Each approximate half of the model disc 105 may include a first wavelength conversion material 30 to convert the source light into the converted light with a red color, a second wavelength conversion material 30 to convert the source light into the converted light with a blue color, and a third wavelength conversion material 30 to convert the source light into the converted light with a green color.

Model disc 106 illustrates a rotatable disc with a plurality of wavelength conversion material 30s located adjacent to approximately nine equal parts of the rotatable disc. The model disc 106 may illustrate a rotatable disc with a plurality of wavelength conversion material 30s located adjacent to approximately two equal thirds of the rotatable disc to alter the level of the affective light to be included in the converted light. A first third of adjacently located wavelength conversion material 30s may increase the level of affective light in the converted light and a third of wavelength conversion material 30s may decrease the level of affective light in the converted light, providing approximately 33% duty cycles between operation in the normal mode, increased mode and decreased mode.

Each approximate third of the model disc 106 may additionally be segmented into three approximately equal parts, with may perform a color conversion with or without altering the level of affective light in the converted light. Each approximate third of the model disc 106 may include a first wavelength conversion material 30 to convert the source light into the converted light with a red color, a second wavelength conversion material 30 to convert the source light into the converted light with a blue color, and a third wavelength conversion material 30 to convert the source light into the converted light with a green color.

The inclusion of a first rotatable disc between the light source, which may be reflected from a repositionable or stationary mirror, and the desired output direction, may allow the source light to be converted into the converted light to include the desired color and levels of the biological affective wavelength range. Additionally, as perhaps best illustrated in FIG. 20, a second rotatable disc may additionally be included between the light source and the desired output direction. The wavelength conversion material 30 located adjacent to the second rotatable disc may provide an additional wavelength conversion operation to supplement the wavelength conversion operation performed by the wavelength conversion material 30 located adjacent to the first rotatable disc. As an example, provided in the interest of clarity and without limitation, the first disc may selectively control the level of altered light in an interim light, while the second disc may perform the color conversion to provide the converted light with a desired output color.

Referring now to FIG. 21, an illustrative configuration of the wavelength adapting device 10 of the present invention will now be discussed. In this example, the source light may be received by a repositionable mirror from a light source. The repositionable mirror may include an adjacently located wavelength conversion material 30 to convert the first level of affective light included in the source light into a second level of affective light to be included in the converted light. The repositionable mirror may be included in an array 55 of mirrors. The array 55 of mirrors may additionally include repositionable mirrors that are not located adjacent to a wavelength conversion material 30. Additionally, the array 55 of repositionable mirrors may be included in a MEMS device.

The source light may be reflected by the repositionable mirror in the direction of a rotatable first disc. As discussed above, the first disc may include a wavelength conversion material 30 located adjacent to at least a part of the disc. Upon receiving the light from the repositionable mirror, which may include light that has undergone a first wavelength conversion, the wavelength conversion material 30 may selectively perform a subsequent wavelength conversion to convert the light into the converted light.

It is also understood that the source light may be received by a mirror from a selectable plurality of light sources. The mirror may be a stationary mirror or a repositionable mirror. The source light may be reflected by the mirror in the desired output direction. One or more light source may include a wavelength conversion material 30 located in line between the light source and the mirror. Upon receiving the source light from the light source, the repositionable mirror may reflect converted light in the desired output direction.

The one or more light sources may additionally be operatively connected to the controller 61, which may control at which source light may emit the source light in the direction of the mirror. More specifically, the controller 61 may selectively control whether to emit source light from a light source absent an intermediary wavelength conversion material 30 in the normal mode or, conversely, to emit source light from a light source with an intermediary wavelength conversion material 30 in the altered mode.

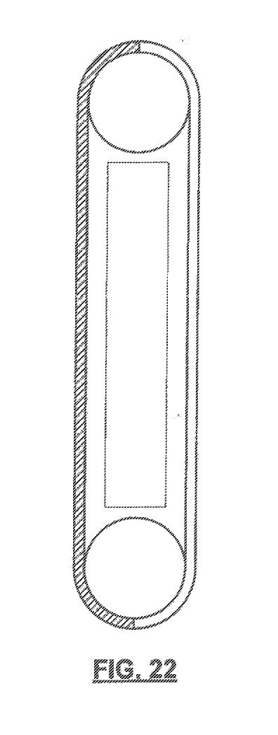

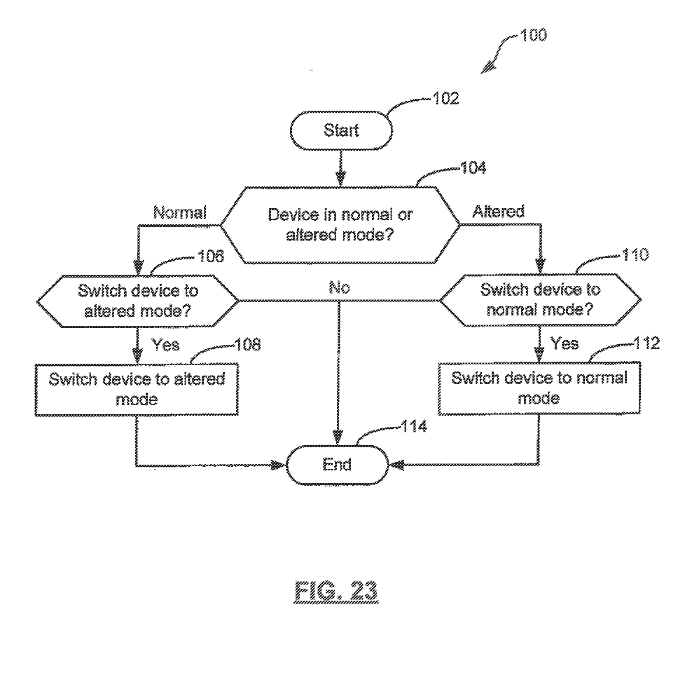

Referring now to FIG. 22, an illustrative configuration of wavelength adapting device 10 of the present invention will now be discussed. In this example, the movable positioning of the wavelength conversion material 30 may occur by cycling a repositionable sheet. The repositionable sheet may include an engaged position to operate in the normal mode, a disengaged position to operate in the altered mode, and any number of intermediary positions. The sheet may be located adjacent to, and be driven by, rollers. A person of skill in the art will appreciate additional operative structures capable of repositioning the sheet to be included within the scope and spirit of the present invention.

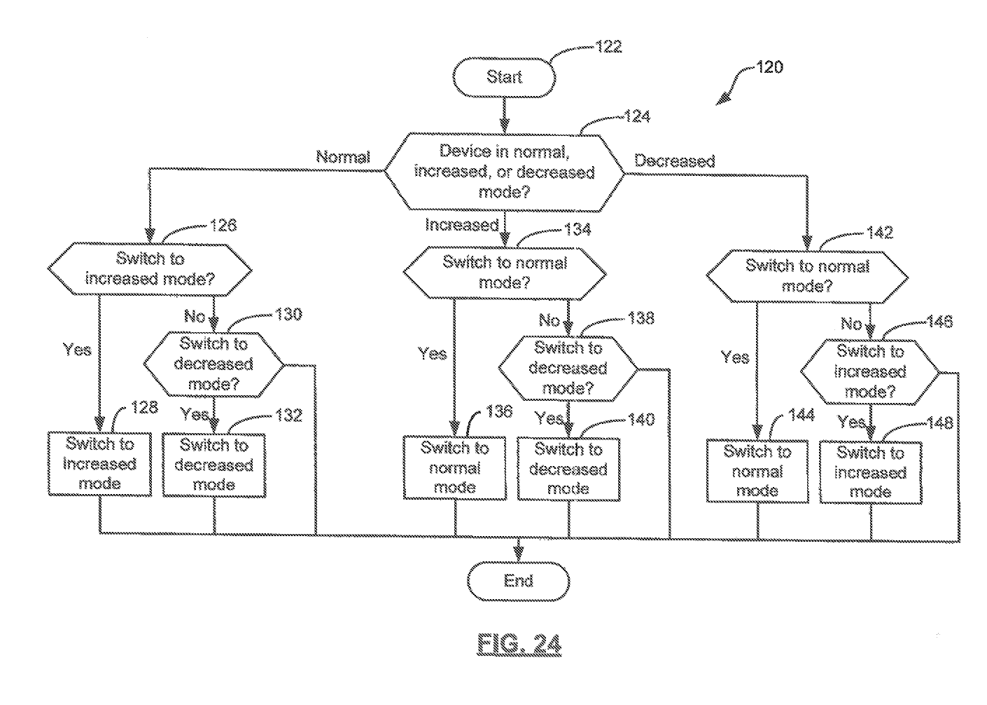

Referring now to the flowchart 100 of FIG. 23, a method for adapting light that includes a source light using a wavelength converting device will now be discussed. The wavelength converting device may include a wavelength conversion material to convert the source light into a converted light to be included generally in the light, and a controller to control operation of the wavelength converting device. Starting at Block 102, a determination may be made of the state of the device, that is, whether it is in a normal mode or an altered mode (Block 104). If the device is in a normal mode, the device may be switchable to an altered mode (Block 106). If the device is to be switched, the wavelength converting device may be operated in an altered mode at Block 108. The process may then end at Block 114. If the device is not to be switched, the process may also end at Block 114. If the device is in an altered mode at Block 104, the device may be switchable to a normal mode (Block 110). If the device is to be switched, the wavelength converting device may be operated in a normal mode at Block 112. The process may then end at Block 114. If the device is not to be switched, the process may end at Block 114.

The source light may includes a first level of affective light within a biological affective wavelength range that may affect a physiological response. The converted light may additionally include a second level of the affective light within the biological affective wavelength range. The normal mode mentioned above may be defined by the second level of the affective light being substantially similar to the first level of the affective light, and the altered mode may be defined by the second level of the affective light differing from the first level of the affective light. The source light including the first level of the affective light may be defined by a first chromaticity, while the converted light including the second level of the affective light may be defined by a second chromaticity. The first chromaticity may be substantially similar to the second chromaticity.

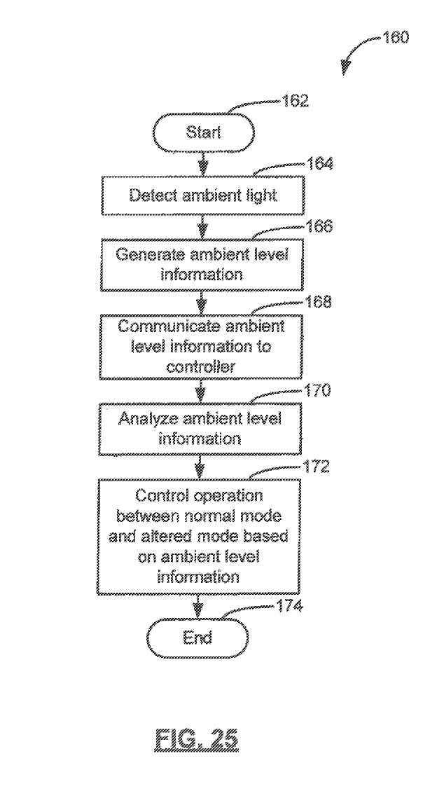

Referring now to flowchart 120 of FIG. 24, an alternate method will be discussed wherein the altered mode may include an increased mode and a decreased mode. The increased mode may be defined by the second level being greater than the first level, and the decreased mode may be defined by the second level being less than the first level. Starting at Block 122, the device's operating mode may be determined (Block 124). If the device is in normal mode, the device may have the option to switch to increased mode at Block 126. If the device is switched to increased mode at Block 128, the process may end at Block 150. If the device is not switched to increased mode, the device may have the option to switch to decreased mode at Block 130. If the device is switched to decreased mode at Block 132, the process may end at Block 150. If the device is not switched to decreased mode, the process may also end at Block 150.

If the device is in increased mode at Block 124, it may have the option to switch to normal mode at Block 134. If the device is switched to normal mode at Block 136, the process may end at Block 150. If the device is not switched to normal mode, the device may be switched to decreased mode at Block 138. If the device is switched to decreased mode at Block 140, the process may end at Block 150. If the device is not switched to decreased mode, the process may also end at Block 150.

If the device is in decreased mode at Block 124, it may have the option to switch to normal mode at Block 142. If the device is switched to normal mode at Block 144, the process may end at Block 150. If the device is not switched to normal mode, the device may be switched to increased mode at Block 146. If the device is switched to increased mode at Block 148, the process may end at Block 150. If the device is not switched to increased mode, the process may also end at Block 150.

Referring now to flowchart 160 of FIG. 25, yet another method of operating the wavelength converting device according to an embodiment of the present invention will be discussed. Starting at Block 162, ambient light may be detected (Block 164). Information regarding the ambient level information may be generated at Block 166. The ambient level information may be communicated to the controller (Block 168), which may analyze ambient level information (Block 170). The operation of the device may be controlled between the normal mode and the altered mode based on the ambient level information (Block 172), ending the method at Block 174.

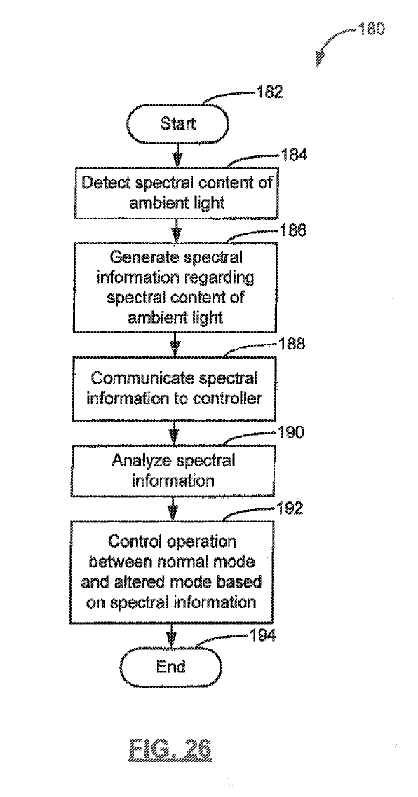

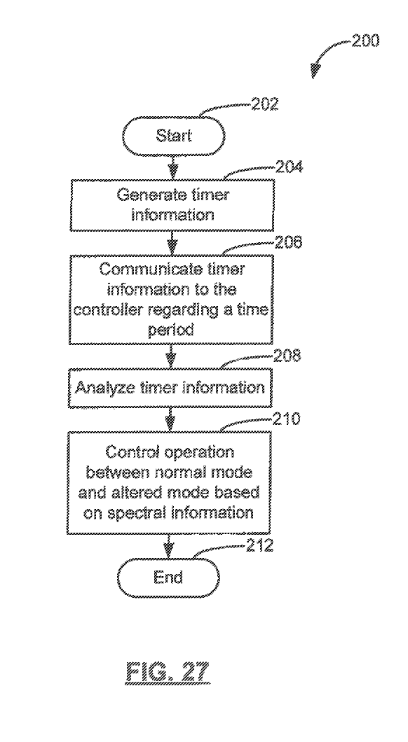

Referring now to flowchart 180 of FIG. 26, yet another method of operating the device according to an embodiment of present invention will be discussed. Starting at Block 182, a spectral content of ambient light may be detected (Block 184). Spectral information regarding the spectral content of the ambient light may be generated at Block 186. The spectral information may be communicated to the controller (Block 188), which may analyze the spectral information (Block 190). The operation of the device may then be controlled between the normal mode and the altered mode based on the spectral information (Block 192), ending the method at Block 194.

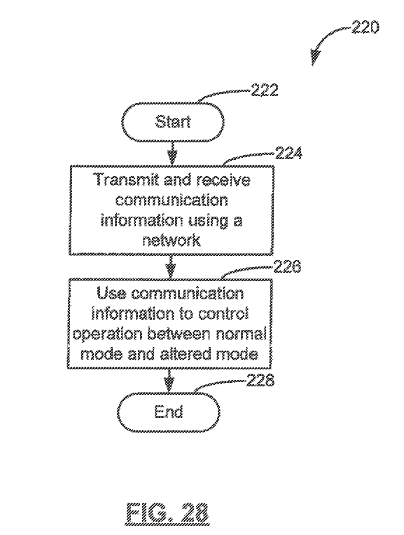

Referring now to flowchart 200 of FIG. 27, still another method of operating the device according to an embodiment of the present invention will be discussed. Starting at Block 202, timer information may be generated (Block 204). The timer information may regard a time period, and may be communicated to the controller (Block 206), which may analyze the timer information (Block 208). The operation of the device may then be controlled between the normal mode and the altered mode based on the timer information (Block 210), ending the method at Block 212).

Referring now to flowchart 220 of FIG. 28, another method of operating the device according to an embodiment of the present invention will be discussed wherein the controller may be communicatively connected to a radio logic board. Starting at Block 222, the method may include transmitting and receiving communication information using a network (Block 224) and using the communication information to control operation between the normal mode and the altered mode (Block 226). The method may end at Block 228.

The methods described above may additionally include controlling brightness of the source light and the converted light using the controller. Additionally, the source light may be received with a display, which may be a liquid crystal display (LCD) that uses color field sequential switching. Such a display may be included in a computerized device.

Many modifications and other embodiments of the invention will come to the mind of one skilled in the art having the benefit of the teachings presented in the foregoing descriptions and the associated drawings. Therefore, it is understood that the invention is not to be limited to the specific embodiments disclosed, and that modifications and embodiments are intended to be included within the scope of the appended claims.

1. A wavelength converting device for adapting light that includes a source light, the wavelength converting device comprising:
a wavelength conversion material to convert the source light into a converted light, wherein the source light includes a first level of affective light within a biological affective wavelength range that affects a physiological response, and wherein the converted light includes a second level of the affective light within the biological affective wavelength range; and
a controller to control operation between a normal mode and an altered mode, wherein the normal mode is defined by the second level of the affective light being similar to the first level of the affective light, and wherein the altered mode is defined by the second level of the affective light differing from the first level of the affective light;
wherein the source light including the first level of the affective light is defined by a first chromaticity;
wherein the converted light including the second level of the affective light is defined by a second chromaticity;
wherein the first chromaticity is a measure of a quality of color of the first level of affective light;
wherein the second chromaticity is a measure of a quality of color of the second level of affective light; and
wherein the first chromaticity is nearly indistinguishable from the second chromaticity.

2. A device according to claim 1:
wherein the altered mode includes an increased mode and a decreased mode;
wherein the increased mode is defined by the second level being greater than the first level; and
wherein the decreased mode is defined by the second level being less than the first level.

3. A device according to claim 1 wherein the physiological response is melatonin production.

4. A device according to claim 1 wherein the source light is emitted from a light source.

5. A device according to claim 4 wherein the light source includes a light emitting diode (LED).

6. A device according to claim 4:
wherein the light source includes a non-affective light source and an affective light source;
wherein the non-affective light source emits the source light with the first level of the affective light;
wherein the affective light source includes a wavelength conversion optic to emit the source light and convert the source light into the converted light with the second level of the affective light; and
wherein the affective light source and the non-affective light source are selectively enabled.

7. A device according to claim 1 wherein the wavelength conversion material is carried by a selectively rotatable disc to enable operation between the normal mode and the altered mode.

8. A device according to claim 7 wherein the rotatable disc includes a plurality of portions; wherein each of the portions correlates with at least one condition; and wherein the rotatable disc is positionable to selectively receive the source light at each portion to manipulate the source light.

9. A device according to claim 8 wherein the at least one condition is selected from the group consisting of color, biological affect, chromaticity, luminosity, saturation, and hue.

10. A device according to claim 1 further comprising a mirror having a light reflective surface to receive and reflect at least one of the source light and the converted light.

11. A device according to claim 10 wherein the wavelength conversion material is located adjacent to at least part of the light reflective surface; wherein the source light is received by the mirror during the altered mode; and wherein the source light is converted by the wavelength conversion material to the converted light with the second level of the affective light to be reflected.

12. A device according to claim 10 wherein the wavelength conversion material is located adjacent to a first part of the reflective surface; wherein no wavelength conversion material is located adjacent to a second part of the reflective surface; wherein the first part of the reflective surface receives and converts the source light to the converted light to be reflected with the second level of affective light; and wherein the second part of the reflective surface receives the source light to be reflected with the first level of affective light.

13. A device according to claim 10 wherein the mirror is included in an array of mirrors; and wherein reflection of at least one of the source light and the converted light from each mirror in the array of mirrors is selectable.

14. A device according to claim 10:
wherein the mirror is a repositionable mirror to be selectively repositioned by the controller;
wherein the repositionable mirror is included in an array of repositionable mirrors;
wherein the wavelength conversion material is located adjacent to at least one repositionable mirror included in the array to receive and convert the source light to the converted light to be reflected with the second level of affective light; and
wherein no conversion material is located adjacent to at least one repositionable mirror included in the array to receive the source light to be reflected with the first level of affective light.

15. A device according to claim 10 wherein the repositionable mirror is included in a microelectromechanical device (MEMS).

16. A device according to claim 1 further comprising a sensor to detect ambient light and generate ambient level information to be communicated to the controller regarding the ambient light; and wherein the controller analyzes the ambient level information to control operation between the normal mode and the altered mode.

17. A device according to claim 1 further comprising a sensor to detect a spectral content of ambient light and generate spectral information to be communicated to the controller regarding the spectral content of the ambient light; and wherein the controller analyzes the spectral information to control operation between the normal mode and the altered mode.

18. A device according to claim 1 further comprising a timer to generate timer information to be communicated to the controller regarding a time period; and wherein the controller analyzes the timer information to control operation between the normal mode and the altered mode.

19. A device according to claim 1 wherein the controller is communicatively connected to a radio logic board to transmit and receive communication information using a network; and wherein the communication information is used by the controller to control operation between the normal mode and the altered mode.

20. A device according to claim 1 wherein the biological affective wavelength range is defined as being essentially between 460 nanometers and 490 nanometers.

21. A device according to claim 1 wherein brightness of the source light and the converted light is controllable by the controller.

22. A device according to claim 1 wherein the source light is received by a display.

23. A device according to claim 22 wherein the display is a liquid crystal display (LCD).

24. A device according to claim 23 wherein the display uses color field sequential switching.

25. A device according to claim 23 wherein the display is included in a computerized device.

26. A method for adapting light that includes a source light using a wavelength converting device that includes a wavelength conversion material to convert the source light into a converted light, and a controller to control operation of the wavelength converting device, the method comprising:
operating the wavelength converting device between a normal mode and an altered mode;
wherein the source light includes a first level of affective light within a biological affective wavelength range that affects a physiological response;
wherein the converted light includes a second level of the affective light within the biological affective wavelength range;
wherein the normal mode is defined by the second level of the affective light being similar to the first level of the affective light;
wherein the altered mode is defined by the second level of the affective light differing from the first level of the affective light;
wherein the source light including the first level of the affective light is defined by a first chromaticity;
wherein the converted light including the second level of the affective light is defined by a second chromaticity;
wherein the first chromaticity is a measure of a quality of the color of the first level of affective light;
wherein the second chromaticity is a measure of a quality of color of the second level of affective light; and
wherein the first chromaticity is nearly indistinguishable from the second chromaticity.

27. A method according to claim 26:
wherein the altered mode includes an increased mode and a decreased mode;
wherein the increased mode is defined by the second level being greater than the first level; and
wherein the decreased mode is defined by the second level being less than the first level.

28. A method according to claim 26 wherein the physiological response is melatonin production; and wherein the source light is emitted from a light source.

29. A method according to claim 28 wherein the light source includes a light emitting diode (LED).

30. A method according to claim 28:
wherein the light source includes a non-affective light source and an affective light source;
wherein the non-affective light source emits the source light with the first level of the affective light;
wherein the affective light source includes a wavelength conversion optic to emit the source light and convert the source light into the converted light with the second level of the affective light; and
wherein the affective light source and the non-affective light source are selectively enabled.

31. A method according to claim 26 wherein the wavelength conversion material is carried by a selectively rotatable disc to enable operation between the normal mode and the altered mode.

32. A method according to claim 31 wherein the rotatable disc includes a plurality of portions; wherein each of the portions correlates with at least one condition; and further comprising positioning the rotatable disc to selectively receive the source light at each portion to manipulate the source light.

33. A method according to claim 32 wherein the at least one condition is selected from the group consisting of color, biological affect, chromaticity, luminosity, saturation, and hue.

34. A method according to claim 26 further comprising receiving and reflecting at least one of the source light and the converted light using a mirror having a light reflective surface.

35. A method according to claim 34 wherein the wavelength conversion material is located adjacent to at least part of the light reflective surface; and further comprising receiving the source light by the mirror during the altered mode; and converting the source light using the wavelength conversion material to the converted light with the second level of the affective light to be reflected.

36. A method according to claim 34 wherein the wavelength conversion material is located adjacent to a first part of the reflective surface; wherein no wavelength conversion material is located adjacent to a second part of the reflective surface; and further comprising receiving and converting the source light to the converted light to be reflected with the second level of affective light using the first part of the reflective surface; and receiving the source light to be reflected with the first level of affective light using the second part of the reflective surface.

37. A method according to claim 34 wherein the mirror is included in an array of mirrors; and wherein reflection of at least one of the source light and the converted light from each mirror in the array of mirrors is selectable.

38. A method according to claim 34:
wherein the mirror is a repositionable mirror to be selectively repositioned by the controller;
wherein the repositionable mirror is included in an array of repositionable mirrors;
wherein the wavelength conversion material is located adjacent to at least one repositionable mirror included in the array to receive and convert the source light to the converted light to be reflected with the second level of affective light; and
wherein no conversion material is located adjacent to at least one repositionable mirror included in the array to receive the source light to be reflected with the first level of affective light.

39. A method according to claim 34 wherein the repositionable mirror is included in a microelectromechanical device (MEMS).

40. A method according to claim 26 further comprising:
detecting ambient light;
generating ambient level information;
communicating the ambient level information to the controller;
analyzing the ambient level information;
controlling operation between the normal mode and the altered mode based on the ambient level information.

41. A method according to claim 26 further comprising:
detecting a spectral content of ambient light;
generating spectral information regarding the spectral content of the ambient light;
communicating the spectral information to the controller;
analyzing the spectral information; and
controlling operation between the normal mode and the altered mode based on the spectral information.

42. A method according to claim 26 further comprising:
generating timer information;
communicating the timer information to the controller regarding a time period;
analyzing the timer information; and
controlling operation between the normal mode and the altered mode based on the timer information.

43. A method according to claim 26 wherein the controller is communicatively connected to a radio logic board; and further comprising:
transmitting and receiving communication information using a network; and
using the communication information to control operation between the normal mode and the altered mode.

44. A method according to claim 26 wherein the biological affective wavelength range is defined as being essentially between 460 nanometers and 490 nanometers.

45. A method according to claim 26 further comprising controlling brightness of the source light and the converted light using the controller.

46. A method according to claim 26 further comprising receiving the source light with a display; wherein the display is a liquid crystal display (LCD); wherein the display uses color field sequential switching; and wherein the display is included in a computerized device.
